(19) United States
(12) Patent Application Publication
(10) Pub. No.: US 2016/0313334 A1
(43) Pub. Date: Oct. 27, 2016

(54) METHODS FOR THE DETECTION OF ESOPHAGEAL ADENOCARCINOMA

(71) Applicant: UNIVERSITY OF PITTSBURGH - OF THE COMMONWEALTH SYSTEM OF HIGHER EDUCATION, Pittsburgh, PA (US)

(72) Inventors: Ali Hussainy Zaidi, Munhall, PA (US); Blair Anderson Jobe, Pittsburgh, PA (US); Xuemei Zeng, Pittsburgh, PA (US); Jeya Balaji Balasubramanian, Pittsburgh, PA (US); Vanathi Gopalakrishnan, Pittsburgh, PA (US); William Lyman Bigbee, San Diego, CA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 15/107,444

(22) PCT Filed: Dec. 29, 2014
(86) PCT No.: PCT/US2014/072538

§ 371 Date: Jun. 22, 2016

Related U.S. Application Data
(60) Provisional application No. 61/922,665, filed on Dec. 31, 2013.

(51) Int. Cl. G01N 33/574; C12Q 1/68

(52) U.S. Cl. CPC G01N 33/57407; C12Q 1/6886; C12Q 2600/158; C12Q 2600/118; G01N 2333/904; G01N 2333/4718; G01N 30/7233

(57) ABSTRACT

Methods are disclosed for detecting the likelihood that a subject will develop esophageal adenocarcinoma. Methods are also disclosed for determining if an agent is effective for treatment or prevention of esophageal adenocarcinoma in a subject. Methods are also disclosed for treating a subject. These methods include detecting a level of biglycan, myeloperoxidase, and protein S100-A9 in a biological sample from the subject administered the agent; and comparing the level of biglycan, myeloperoxidase, and protein S100-A9 to a respective control level of biglycan, myeloperoxidase, and protein S100-A9. In some embodiments, these methods also include detecting a level of annexin-A6 in a biological sample from the subject; and comparing the level of annexin-A6 to a respective control level of annexin-A6.

### CROSS REFERENCE TO RELATED APPLICATION(S)

This claims the benefit of U.S. Provisional Application No. 61/922,665, filed Dec. 31, 2013, which is incorporated by reference herein.

### ACKNOWLEDGMENT OF GOVERNMENT SUPPORT

This invention was made with government support under LM010950 awarded by the National Institutes of Health. The government has certain rights in the invention.

### FIELD

This is related to the field of esophageal adenocarcinoma, specifically to personalized medicine and methods for detecting esophageal adenocarcinoma.

### BACKGROUND

The incidence of esophageal adenocarcinoma (EAC) is rapidly rising; outpacing the rate of increase of all other cancers. The number of patients affected is up to 600% higher than in the 1970s' (Dubecz et al., J Gastrointest Surg. 2013 Nov. 15; Prasad et al., Amer. J. Gastroentero. 2010; 105(7):1490-502). Additionally, EAC is associated with a dismal prognosis, with a five-year survival of less than 15%. Although the survival and prognosis depends on the stage of the disease, unfortunately, because the esophagus is a distensible organ, the majority of patients who develop EAC do not sense difficulty swallowing until the tumor is fairly advanced (Zhang, World J Gastroenterol. 2013 Sep. 14; 19(34):5598-606). There is an urgent need for improving risk stratification to facilitate early detection and thereby reducing the mortality due to EAC.

Currently without clinical risk factors that signal the early development of EAC (Spechler, JAMA. 2013 Aug. 14; 310(6):627-36); the identification of early-stage and curable disease is only possible through endoscopic Barrett's esophagus (BE) screening in patients with symptoms of gastroesophageal reflux disease (GERD) (Spechler, Amer. J. Med. 2001; 111 Suppl. 8A:130S-6S; Sampliner, American J Gasteroenterol. 1998; 93(7):1028-32). Those diagnosed with BE then typically undergo lifetime endoscopic surveillance for the development of malignancy (Sampliner, Amer. J. Gastroenterol. 2002; 97(8):1888-95). However, 95% of patients who develop EAC have never undergone BE screening prior to cancer diagnosis and up to 57% of patients who develop EAC do not report antecedent GERD symptoms (see Conio et all, Gut. 2001; 48(3):304-9; Corley et al., Gastroenterol. 2002; 122(3):633-40; Dulai et al., Gastroenterology. 2002; 122(1):26-33. Epub 2002/01/10. PubMed PMID: 11781277; Lagergren et al., NEJM. 1999; 340(11):825-31; Reavis et al., Annal. Surg. 2004; 239(6):849-56).

The identification of cancer biomarkers raises the possibility for early detection, better monitoring of tumor progression and/or response to therapy. Protein biomarkers that have been identified and are in regular clinical use for other types of tumors include carcinoembryonic antigen (CEA), prostate specific antigen (PSA), alpha-fetoprotein (AFP) and cancer antigen 125 (CA-125). The development of biomarkers is even more important for cancers like EAC that are typically diagnosed at advanced stages of disease and have poor long-term survival rates with the currently employed management paradigm (Reid et al., Am J Gastroenterol. 2001 October; 96(10):2839-48; Kato et al., Int J Cancer. 2001 Mar. 20; 95(2):92-5). The development of such biomarkers also can be important to help select patients in whom the risk of esophageal endoscopy is justified, or in whom chronic acid suppressive therapy is indicated, for example with proton pump inhibitors. Thus, a need remains for biomarkers that can be used to determine the likelihood that a subject will develop EAC and/or determine if an agent is effective for treating EAC.

### SUMMARY

Methods are disclosed for detecting the likelihood that a subject will develop EAC or is to be subjected to further diagnostic investigation, such as, but not limited to, clinical procedures such as endoscopic examination of the esophagus. These methods include detecting a level of biglycan, myeloperoxidase, and protein S100-A9 in a biological sample from the subject; and comparing the level of biglycan, myeloperoxidase, and protein S100-A9 to a respective control level of biglycan, myeloperoxidase, and protein S100-A9. Detection of an increase in the level of biglycan, myeloperoxidase, and protein S100-A9 as compared to the respective control indicates the likelihood that the subject has or will develop EAC.

In some embodiments, these methods include performing an assay that detects a level of annexin-A6 in a biological sample from the subject; and comparing the level of annexin-A6 to a respective control level of annexin-A6. The detection of increase in the level of annexin-A6 as compared to the respective control level of annexin-A6 indicates the likelihood that the subject has or will develop EAC.

Methods are also disclosed for determining if an agent is effective for treatment or prevention of EAC in a subject. These methods include detecting a level of biglycan, myeloperoxidase, and protein S100-A9 in a biological sample from the subject administered the agent; and comparing the level of biglycan, myeloperoxidase, and protein S100-A9 to a respective control level of biglycan, myeloperoxidase, and protein S100-A9. The detection of a decrease in the level of biglycan, myeloperoxidase, and protein S100-A9 compared to the respective control indicates the likelihood that the agent is effective for the treatment or prevention of EAC the subject.

In some embodiments, these methods include detecting a level of annexin-A6 in a biological sample from the subject; and comparing the level of annexin-A6 to a respective control level of annexin-A6. The detection of a decrease in the level of annexin-A6 as compared to the respective control level of annexin-A6 indicates the likelihood that the pharmaceutical agent is effective for the treatment or prevention of EAC in the subject.

In additional embodiments, the biomarkers are used to select subject for further diagnostic and/or therapeutic interventions. In specific non-limiting examples, a diagnostic intervention includes esophageal endoscopy. In other non-limiting examples, the therapeutic intervention includes surgery, radiation, chemotherapy, and/or a pharmaceutical agent.

The foregoing and other objects, features, and advantages of the invention will become more apparent from the following detailed description, which proceeds with reference to the accompanying figures.

### BRIEF DESCRIPTION OF THE DRAWINGS

FIG. 1. Study schema with the patient populations and methods used. Serum protein biomarker discovery was guided by tissue-based proteomics followed by analysis and evaluation in serum samples using ELISAs.

FIG. 2. A heat map representation from a supervised clustering analysis of significantly differentially abundant proteins identified from BE in column grouping 1 (n=1.0), HGD in column grouping 2 (n=11) and EAC tissues in column grouping 3 (n=10). Individually significant proteins are represented in row groups 4, 5 and 6. Protein abundances are plotted as mean observed spectral counts for each tissue type where red represents proteins with a normalized spectral count value greater than 1.5 and green represents values less than 1.5. Significance was determined by the Kruskal-Wallis test. The results demonstrate clear patterns of protein abundance that can be seen correlating with BE (nodes 1&4), HGD (nodes 2&5) and EAC (nodes 3&6).

FIGS. 3A-3E. Boxplot distributions of candidate protein abundance in serum samples of EAC vs. GERD patients using ELISAs. Scatter plots are overlaid on top of the box plots to visualize the individual data points [ANXA6 (dilution factor (df)=1600×), BGN (df=200×), S100A9 (df=25×), MPO (df=10×) and resistin (df=5×). For each candidate serum biomarker, on the left are results from the discovery set comprising 20 GERD and 12 EAC samples and on the right are results from the validation set consisting of 36 GERD and 31 EAC samples, respectively. The bottom and top of the boxplots indicate the first and third quartiles of the data, the line within each boxplot is the median value. The length of the boxplot whiskers is specified as 1.5 times the (25^{th} to 75^{th}) interquartile range of the data. For the candidate biomarkers, t-test was used to compare the means of EAC vs. GERD in each set, with a p value<0.05 considered significant. All markers except resistin were significant.

FIG. 4. Visual representation of the final BRL rule model derived from the merged serum ELISA data.

FIG. 5. ROC curve generated from ten-fold cross fold validation on the merged datasets to estimate the classification performance of the final BRL predictive model.

### SEQUENCE LISTING

GENBANK® Accession numbers are provided below. In these entries nucleic and amino acid sequences listed are shown using standard letter abbreviations for nucleotide bases, and one letter code for amino acids, as defined in 37 C.F.R. 1.822. Only one strand of each nucleic acid sequence is shown, but the complementary strand is understood as included by any reference to the displayed strand. All of the GENBARNK® entries are incorporated herein by reference as available on Dec. 1, 2013. The Sequence Listing is submitted as an ASCII text file [92271-02_Sequence.txt, Nov. 13, 2014, 49.1 KB], which is incorporated by reference herein.

### DETAILED DESCRIPTION

Methods are disclosed for detecting the likelihood that a subject will develop EAC. The methods can stratify the risk of developing EAC. Methods are also disclosed for determining if an agent is effective for treatment or prevention of EAC in a subject.

### TERMS

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. All GENBANK® Accession Nos. listed herein are incorporated by reference in their entirety. Although methods and materials similar or equivalent to those described herein can be used in the practice or testing of the present disclosure, suitable methods and materials are described below. The materials, methods, and examples are illustrative only and not intended to be limiting.

Alter: A change in an effective amount of a substance of interest, such as a polypeptide, for example, of biglycan, myeloperoxidase, protein S100-A9 or annexin-A6, or a polynucleotide encoding the polypeptide. The amount of the substance can changed by a difference in the amount of the substance produced, by a difference in the amount of the substance that has a desired function, or by a difference in the activation of the substance. The change can be an increase or a decrease. The alteration can be in vivo or in vitro.

In several embodiments, altering an amount of a polypeptide or polynucleotide is at least about a 50%, 60%, 70%, 80%, 90%, 95%, 96%, 97%, 98%, 99%, or 100% increase or decrease in the effective amount (level) of a substance, such as, but not limited to, biglycan, myeloperoxidase, protein S100-A9 or annexin-A6. In specific example, an increase of a polypeptide or polynucleotide is at least about a 50%, 60%, 70%, 80%, 90%, 95%, 96%, 97%, 98%, 99%, or 100% increase in a substance, such as, but not limited to, biglycan, myeloperoxidase, protein S100-A9 or annexin-A6 polypeptide, or a polynucleotide encoding the polypeptide, as compared to a control, a statistical normal, or a standard value chosen for specific study. In another specific example, a decrease of a polypeptide or polynucleotide, such as following the initiation of a therapeutic protocol, is at least about a 50%, 60%, 70%, 80%, 90%, 95%, 96%, 97%, 98%, 99%, or 100% decrease in a substance, such as biglycan, myeloperoxidase, protein S100-A9 or annexin-A6 polypeptide, or polynucleotide encoding the polypeptide, as compared to a control, a statistical normal, or a standard value chosen for specific study.

Annexin-A6: A member of a family of calcium-dependent membrane and phospholipid binding proteins. The annexin VI gene is approximately 60 kbp long and contains 26 exons. It encodes a protein of about 68 kDa that consists of eight 68-amino acid repeats separated by linking sequences of variable lengths. It is highly similar to human annexins I and II sequences, each of which contain four such repeats.

Antibody: A polypeptide including at least a light chain or heavy chain immunoglobulin variable region which specifically recognizes and binds an epitope of an antigen or an antigen-binding fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for the molecules listed, such as biglycan, myeloperoxidase, protein S100-A9 or annexin-A6.

The term antibody includes intact immunoglobulins, as well the variants and portions thereof, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA, and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.”

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species, such as human, and CDRs (which generally confer antigen binding) from another species, such as a murine antibody that specifically binds an antigen of interest.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one example, all the CDRs are from the donor immunoglobulin in a humanized immunoglobulin. Constant regions need not be present, but if they are, they are substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized immunoglobulins can be constructed by means of genetic engineering (see for example, U.S. Pat. No. 5,585,089). A “human” antibody includes human framework regions and CDRs from a human immunoglobulin.

Array: An arrangement of molecules, such as biological macromolecules (such as peptides, antibodies or nucleic acid molecules) or biological samples (such as tissue sections), in addressable locations on or in a substrate. A “microarray” is an array that is miniaturized so as to require or be aided by microscopic examination for evaluation or analysis. Arrays are sometimes called chips or biochips.

The array of molecules (“features”) makes it possible to carry out a very large number of analyses on a sample at one time. In certain example arrays, one or more molecules (such as an oligonucleotide probe) will occur on the array a plurality of times (such as twice), for instance to provide internal controls. The number of addressable locations on the array can vary, for example from at least one, to at least 2, to at least 5, to at least 10, at least 20, at least 30, at least 50, at least 75, at least 100, at least 150, at least 200, at least 300, at least 500, least 550, at least 600, at least 800, at least 1000, at least 10,000, or more. In particular examples, an array includes nucleic acid molecules, such as oligonucleotide sequences that are at least 15 nucleotides in length, such as about 15-40 nucleotides in length. In particular examples, an array includes oligonucleotide probes or primers which can be used to detect EAC.

Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples could be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Protein-based arrays include probe molecules that are or include proteins, or where the target molecules are or include proteins, and arrays including antibodies to which proteins are bound, or vice versa. In some examples, an array contains antibodies to biglycan, myeloperoxidase, protein S100-A9 and/or or annexin-A6.

In some examples, the array includes positive controls, negative controls, or both, for example molecules specific for detecting β-actin, 18S RNA, beta-microglobulin, glyceraldehyde-3-phosphate-dehydrogenase (GAPDH), and other housekeeping genes. In one example, the array includes 1 to 20 controls, such as 1 to 10 or 1 to 5 controls.

Barrett's Esophagus: An abnormal change (metaplasia) in the cells of the lower portion of the esophagus. Barrett's esophagus is the diagnosis when the normal stratified squamous epithelium lining of the esophagus is replaced by simple columnar epithelium with goblet cells. Barrett's esophagus is found in 5-15% of patients who seek medical care for gastroesophageal reflux disease (GERD), although a large subgroup of patients with Barrett esophagus do not have symptoms. Barrett's esophagus is its strongly association with esophageal adenocarcinoma, and is considered to be a premalignant condition. The main cause of Barrett's esophagus is thought to be an adaptation to chronic acid exposure from reflux esophagitis. The cells of Barrett's esophagus, after biopsy, are classified into four general categories: non-dysplastic, low-grade dysplasia, high-grade dysplasia, and frank carcinoma.

Biglycan: A small leucine-rich repeat proteoglycan (SLRP) which in vivo is found in a variety of extracellular matrix tissues, including bone, cartilage and tendon. Biglycan consists of a biglycan protein core containing leucine-rich repeat regions, and in vivo has two glycosaminoglycan (GAG) chains consisting of either chondroitin sulfate (CS) or dermatan sulfate (DS), with DS being more abundant in most connective tissues. The CS/DS chains are attached at amino acids 5 and 10 in human biglycan.

Consists essentially of: In the context of the present disclosure, “consists essentially of” indicates that the expression of additional markers associated with a disorder can be evaluated, but not more than ten additional associated markers. In some examples, “consists essentially of” indicates that no more than 5 other molecules are evaluated, such as no more than 4, 3, 2, or 1 other molecules. In some examples, the expression of one or more controls is evaluated, such as a housekeeping protein or rRNA (such as 18S RNA, beta-microglobulin, GAPDH, and/or β-actin) in addition to the genes associated with the disorder. In this context “consists of” indicates that only the expression of the stated molecules is evaluated; the expression of additional molecules is not evaluated.

Control: A “control” refers to a sample or standard used for comparison with an experimental sample. In some embodiments, the control is a sample obtained from a healthy patient or a non-diseased tissue sample obtained from a patient diagnosed with the disorder of interest, such as EAC. In some embodiments, the control is a historical control or standard reference value or range of values (such as a previously tested control sample, such as a group of patients with the disorder, or group of samples that represent baseline or normal values, such as the level of specific genes in non-diseased tissue).

Detecting expression of a gene product: Determining the presence of and/or the level of expression of a nucleic acid molecule (such as an mRNA molecule) or a protein encoded by a gene in either a qualitative or quantitative manner. Exemplary methods include microarray analysis, RT-PCR, Northern blot, Western blot, and mass spectrometry of specimens from a subject, for example measuring levels of a gene product present in blood, serum, or another biological sample as a measure of expression.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Differential or alteration in expression: A difference or change, such as an increase or decrease, in the conversion of the information encoded in a gene into messenger RNA, the conversion of mRNA to a protein, or both. In some examples, the difference is relative to a control or reference value or range of values, such as an amount of gene expression that is expected in a subject who does not have a disorder of interest (for example esophageal adenocarcinoma, Barrett's esophagus, high grade dysplasia or low grade dysplasia of the esophagus). Detecting differential expression can include measuring a change in gene expression or a change in protein levels.

Downregulated or decreased: When used in reference to the expression of a nucleic acid molecule, such as a gene, refers to any process which results in a decrease in production of a gene product, such as a protein. A gene product can be RNA (such as microRNA, mRNA, rRNA, tRNA, and structural RNA) or protein. Therefore, gene downregulation or deactivation includes processes that decrease transcription of a gene or translation of mRNA.

Examples of processes that decrease transcription include those that facilitate degradation of a transcription initiation complex, those that decrease transcription initiation rate, those that decrease transcription elongation rate, those that decrease processivity of transcription and those that increase transcriptional repression. Gene downregulation can include reduction of expression above an existing level. Examples of processes that decrease translation include those that decrease translational initiation, those that decrease translational elongation and those that decrease mRNA stability.

Gene downregulation includes any detectable decrease in the production of a gene product. In certain examples, production of a gene product decreases by at least 2-fold, for example at least 3-fold or at least 4-fold, as compared to a control (such an amount of gene expression in a normal cell). In one example, a control is a relative amount of gene expression in a biological sample, such as from a subject that does not have ASCVD or has not had an MI.

Expression: The process by which the coded information of a gene is converted into an operational, non-operational, or structural part of a cell, such as the synthesis of a protein. Gene expression can be influenced by external signals. Different types of cells can respond differently to an identical signal. Expression of a gene also can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced. In an example, gene expression can be monitored to determine the diagnosis and/or prognosis of a subject with EAC.

The expression of a nucleic acid molecule in a test sample can be altered relative to a control sample, such as a normal sample from a healthy subject. Expression of proteins is the level of protein in a biological sample. Expression includes, but is not limited to, the production of the protein by translation of an mRNA and the half-life of the protein. Protein expression can also be altered in some manner to be different from the expression of the protein in a normal (e.g., non-disease) situation. Alterations in expression, such as differential expression, include but are not limited to: (1) overexpression; (2) underexpression; or (3) suppression of expression.

Controls or standards for comparison to a sample, for the determination of differential expression, include samples believed to be normal (in that they are not altered for the desired characteristic, for example a sample from a subject who does not have EAC and/or Barrett's esophagus) as well as laboratory values (e.g., range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values.

Esophagogastroduodenoscopy (EGD) or Upper Gastrointestinal Endoscopy: A diagnostic endoscopic procedure that visualizes any upper part of the gastrointestinal tract up to the duodenum. An “esophageal endoscopy” is any endoscopic procedure that visualizes the esophagus. An esophageal endoscopy may sometimes be performed as part of aan EGD or upper gastrointestinal endoscopy. The terms are not mutually exclusive unless expressly stated to be so.

Gene expression profile (or signature): Differential or altered gene expression can be detected by changes in the detectable amount of gene expression (such as cDNA or mRNA) or by changes in the detectable amount of proteins expressed by those genes. A distinct or identifiable pattern of gene expression, for instance a pattern of high and low expression of a defined set of genes or gene-indicative nucleic acids such as ESTs. A gene expression profile (also referred to as a signature) can be linked to disease progression (such as advanced EAC), or to any other distinct or identifiable condition that influences gene expression in a predictable way. Gene expression profiles can include relative as well as absolute expression levels of specific genes, and can be viewed in the context of a test sample compared to a baseline or control sample profile (such as a sample from the same tissue type from a subject who does not have EAC). In one example, a gene expression profile in a subject is read on an array (such as a nucleic acid or protein array). For example, a gene expression profile can be performed using a commercially available array such as Human Genome GENECHIP® arrays from AFFYMETRIX® (Santa Clara, Calif.). Typical symptoms are heartburn and regurgitation. Less common symptoms are pain with swallowing, increased salivation, nausea, chest pain and coughing.

Gastroesophageal Reflux Disease (GERD): A chronic symptom of mucosal damage caused by stomach acid coming up from the stomach into the esophagus. GERD is usually caused by changes in the barrier between the stomach and the esophagus, including abnormal relaxation of the lower esophageal sphincter, which normally holds the top of the stomach closed, impaired expulsion of gastric reflux from the esophagus, or a hiatal hernia. These changes may be permanent or temporary.

Hybridization: To form base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the Na concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 60% Identity)

Hybridization: 6×SSC at RT to 55° C. for 16-20 hours

Wash at least twice: 2×-3×SSC at RT to 55° C. for 20-30 minutes each

Isolated: An “isolated” biological component (such as a nucleic acid molecule, protein, or cell) has been substantially separated or purified away from other biological components in the cell of the organism, or the organism itself, in which the component naturally occurs, such as other chromosomal and extra-chromosomal DNA and RNA, proteins and cells. Nucleic acid molecules and proteins that have been “isolated” include nucleic acid molecules and proteins purified by standard purification methods. The term also embraces nucleic acid molecules and proteins prepared by recombinant expression in a host cell as well as chemically synthesized nucleic acid molecules and proteins.

Label: An agent capable of detection, for example by ELISA, spectrophotometry, flow cytometry, or microscopy. For example, a label can be attached to a nucleic acid molecule or protein, thereby permitting detection of the nucleic acid molecule or protein. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998). In a particular example, a label is conjugated to a binding agent that specifically binds to a protein of interest, such as biglycan, myeloperoxidase, protein S-100-A9 or annexin-A6, as disclosed herein.

Level of Expression: An amount, such as of a protein or an mRNA, that can be measured in a biological sample.

Low Grade Dysplasia and High Grade Dysplasia (of the Esophagus): Pathological conditions of the esophagus. Generally, in dysplasia there is an absence of apical mucin the espophagus. Frequently, both an absence of goblet cells and mucin depletion in the non-goblet columnar cells are seen in dysplastic epithelium. At low power, these areas appear more hyperchromatic as compared to uninvolved areas.

For high grade dysplasia, distortion of glandular architecture of the espophagus is usually is present and may be marked; it is composed of branching and lateral budding of crypts, a villiform configuration of the mucosal surface, or intraglandular bridging of epithelium to form a cribriform pattern of “back-to-back” glands. There is dysplastic epithelium on the mucosal surface with loss of nuclear polarity, characterized by “rounding up” of the nuclei, and absence of a consistent relationship of nuclei to each other.

Mammal: This term includes both human and non-human mammals. Examples of mammals include, but are not limited to: humans, pigs, cows, goats, cats, dogs, rabbits, rats, and mice.

Mass Spectrometry: A process used to separate and identify molecules based on their mass. Mass spectrometry ionizes chemical compounds to generate charged molecules or molecule fragments and measures their mass-to-charge ratios. In a typical MS procedure, as sample is ionized. The ions are separated according to their mass-to-charge ratio, and the ions are dynamically detected by some mechanism capable of detecting energetic charged particles. The signal is processed into the spectra of the masses of the particles of that sample. The elements or molecules are identified by correlating known masses by the identified masses. “Time-of-flight mass spectrometry” (TOFMS) is a method of mass spectrometry in which an ion's mass-to-charge ratio is determined via a time measurement. Ions are accelerated by an electric field of known strength. This acceleration results in an ion having the same kinetic energy as any other ion that has the same charge. The velocity of the ion depends on the mass-to-charge ratio. The time that it subsequently takes for the particle to reach a detector at a known distance is measured. This time will depend on the mass-to-charge ratio of the particle (heavier particles reach lower speeds). From this time and the known experimental parameters one can find the mass-to-charge ratio of the ion. “Liquid chromatography-mass spectrometry” or “LC-MS” is a chemistry technique that combines the physical separation capabilities of liquid chromatography (or HPLC) with the mass analysis capabilities of mass spectrometry. Liquid chromatography mass spectrometry (LC-MS) separates compounds chromatographically before they are introduced to the ion source and mass spectrometer. It differs from gas chromatography (GC-MS) in that the mobile phase is liquid, usually a mixture of water and organic solvents, instead of gas and the ions fragments. Most commonly, an electrospray ionization source is used in LC-MS.

Multiple reaction monitoring (MRM): A mass spectrometry based method in which absolute quantification of a targeted protein(s) can be obtained. In this method external or internal standards are used. Often a known quantity of a synthetic stable isotopically labeled peptide matching each of the targeted peptides that represent unique the protein is added into each sample being quantified. Comparison of the peak of the endogenous peptide to the labeled standard peptide allows absolute quantitation. MRM can be multiplexed easily, allowing multiple phosphorylation sites and/or multiple proteins to be assessed simultaneously.

Myeloperoxidase (MPO): A peroxidase enzyme that is encoded by the MPO gene. MPO is a lysosomal protein stored in azurophilic granules of the neutrophil. The 150-kDa MPO protein is a dimer consisting of two 15-kDa light chains and two variable-weight glycosylated heavy chains bound to a prosthetic heme group.

Nucleic acid array: An arrangement of nucleic acids (such as DNA or RNA) in assigned locations on a matrix, such as that found in cDNA arrays, or oligonucleotide arrays.

Nucleic acid molecules representing genes: Any nucleic acid, for example DNA (intron or exon or both), cDNA, or RNA (such as mRNA), of any length suitable for use as a probe or other indicator molecule, and that is informative about the corresponding gene, such the proteins specified herein.

Peptide/Protein/Polypeptide: All of these terms refer to a polymer of amino acids and/or amino acid analogs that are joined by peptide bonds or peptide bond mimetics, regardless of length or post-translational modification (such as glycosylation, methylation, ubiquitination, phosphorylation, or the like).

Polymerase Chain Reaction (PCR): An in vitro amplification technique that increases the number of copies of a nucleic acid molecule (for example, a nucleic acid molecule in a sample or specimen). The product of a PCR can be characterized by standard techniques known in the art, such as electrophoresis, restriction endonuclease cleavage patterns, oligonucleotide hybridization or ligation, and/or nucleic acid sequencing.

In some examples, PCR utilizes primers, for example, DNA oligonucleotides 10-100 nucleotides in length, such as about 15, 20, 25, 30 or 50 nucleotides or more in length (such as primers that can be annealed to a complementary target DNA strand by nucleic acid hybridization to form a hybrid between the primer and the target DNA strand. Primers can be selected that include at least 15, at least 20, at least 25, at least 30, at least 35, at least 40, at least 45, at least 50 or more consecutive nucleotides of a nucleotide sequence of interest. Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols, A Guide to Methods and Applications, Academic Press, Inc., San Diego, Calif., 1990).

Primers: Short nucleic acid molecules, for instance DNA oligonucleotides 10-100 nucleotides in length, such as about 15, 20, 25, 30 or 50 nucleotides or more in length, such as this number of contiguous nucleotides of a nucleotide sequence encoding a protein of interest or other nucleic acid molecule. Primers can be annealed to a complementary target DNA strand by nucleic acid hybridization to form a hybrid between the primer and the target DNA strand. Primer pairs can be used for amplification of a nucleic acid sequence, such as by PCR or other nucleic acid amplification methods known in the art.

Methods for preparing and using nucleic acid primers are described, for example, in Sambrook et al. (In Molecular Cloning: A Laboratory Manual, CSHL, New York, 1989), Ausubel et al. (In Current Protocols in Molecular Biology, John Wiley & Sons, New York, 1998), and Innis et al. (PCR Protocols, A Guide to Methods and Applications, Academic Press, Inc., San Diego, Calif., 1990). PCR primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular primer increases with its length.

In one example, a primer includes at least 15 consecutive nucleotides of a nucleotide molecule, such as at least 18 consecutive nucleotides, at least 20, at least 25, at least 30, at least 35, at least 40, at least 45, at least 50 or more consecutive nucleotides of a nucleotide sequence (such as a gene, mRNA or cDNA). Such primers can be used to amplify a nucleotide sequence of interest, for example using PCR, such as biglycan, myeloperoxidase, protein S100-A9 and annexin-A6.

Probe: A short sequence of nucleotides, such as at least 8, at least 10, at least 15, at least 20, at least 21, at least 25, at least 30, at least 35, at least 40, at least 45, at least 50, at least 55, at least 60, at least 65, at least 70, at least 75, at least 80, at least 85, at least 90, at least 95 or even greater than 100 nucleotides in length, used to detect the presence of a complementary sequence by molecular hybridization. In particular examples, oligonucleotide probes include a label that permits detection of oligonucleotide probe:target sequence hybridization complexes. Such an oligonucleotide probe can also be used on a nucleic acid array, for example to detect a nucleic acid molecule in a biological sample contacted to the array. In some examples, a probe is used to detect the presence of a nucleic acid molecule for such as biglycan, myeloperoxidase, protein S100-A9 and annexin-A6.

Prognosis: A prediction of the future course of a disease, such as EAC. The prediction can include determining the likelihood of a subject to develop complications of EAC, or to survive a particular amount of time (e.g., determine the likelihood that a subject will survive 1, 2, 3 or 5 years), to respond to a particular therapy (e.g., radiation or chemotherapy), or combinations thereof.

Protein S100-A9: A protein also known as migration inhibitory factor-related protein 14 (MRP-14) or calgranulin-B. This protein is a member of the S100 family of proteins containing 2 EF hand calcium-binding motifs. S100 proteins are localized in the cytoplasm and/or nucleus of a wide range of cells, and involved in the regulation of a number of cellular processes such as cell cycle progression and differentiation. S100 genes include at least 13 members which are located as a cluster on chromosome 1q21.

Purified: The term “purified” does not require absolute purity; rather, it is intended as a relative term. Thus, for example, a purified protein preparation is one in which the protein referred to is more pure than the protein in its natural environment within a cell. For example, a preparation of a protein is purified such that the protein represents at least 50% of the total protein content of the preparation. Similarly, a purified oligonucleotide preparation is one in which the oligonucleotide is more pure than in an environment including a complex mixture of oligonucleotides.

Sample (or biological sample): A biological specimen containing genomic DNA, RNA (including mRNA), protein, or combinations thereof, obtained from a subject. Examples include, but are not limited to, peripheral blood, serum, plasma, urine, fine needle aspirate, tissue biopsy, surgical specimen, and autopsy material.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs may use SEG filtering (Wootton and Federhen, Meth. Enzymol. 266:554-571, 1996). In addition, a manual alignment can be performed. Proteins with even greater similarity will show increasing percentage identities when assessed by this method, such as at least about 75%, 80%, 85%, 90%, 95%, 98%, or 99% sequence identity to biglycan, myeloperoxidase, protein S100-A9, or annexin-A6.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to biglycan, myeloperoxidase, protein S100-A9 or annexin-A6. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity to biglycan, myeloperoxidase, protein S100-A9 or annexin-A6. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Specific Binding Agent: An agent that binds substantially or preferentially only to a defined target such as a protein, enzyme, polysaccharide, oligonucleotide, DNA, RNA, recombinant vector or a small molecule. Thus, a nucleic acid-specific binding agent binds substantially only to the defined nucleic acid, such as RNA, or to a specific region within the nucleic acid. For example, a “specific binding agent” includes an antisense compound (such as an antisense oligonucleotide, siRNA, miRNA, shRNA or ribozyme) that binds substantially to a specified RNA.

A protein-specific binding agent binds substantially only the defined protein, or to a specific region within the protein. For example, a “specific binding agent” includes antibodies and other agents that bind substantially to a specified polypeptide. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Subject: Living multi-cellular vertebrate organism, a category that includes human and non-human mammals.

Therapeutically effective amount: An amount of a pharmaceutical preparation that alone, or together with a pharmaceutically acceptable carrier or one or more additional therapeutic agents, induces the desired response. A therapeutic agent, such as a chemotherapeutic agent or radiation, is administered in therapeutically effective amounts.

Effective amounts a therapeutic agent can be determined in many different ways, such as assaying for a reduction in metastasis, tumor volume, or improvement of physiological condition of a subject, such as a decrease in symptoms, such as difficulty swallowing. Effective amounts also can be determined through various in vitro, in vivo or in situ assays.

Therapeutic agents can be administered in a single dose, or in several doses, for example daily, during a course of treatment. However, the effective amount of can be dependent on the source applied, the subject being treated, the severity and type of the condition being treated, and the manner of administration.

In one example, it is an amount sufficient to partially or completely alleviate symptoms of vascular disease within a subject. Treatment can involve only slowing the progression of the vascular disease temporarily, but can also include halting or reversing the progression of the vascular disease permanently. For example, a pharmaceutical preparation can decrease one or more symptoms of vascular disease, for example decrease a symptom by at least 20%, at least 50%, at least 70%, at least 90%, at least 98%, or even at least 100%, as compared to an amount in the absence of the pharmaceutical preparation.

Translation: The process in which cellular ribosomes create proteins. In translation, messenger RNA (mRNA) produced by transcription is decoded by a ribosome complex to produce a specific polypeptide.

Treating a disease: “Treatment” refers to a therapeutic intervention that ameliorates a sign or symptom of a disease or pathological condition, such a sign, parameter or symptom of EAC. Treatment can also induce remission or cure of a condition, such as EAC. In particular examples, treatment includes preventing a disease, for example by inhibiting the full development of a disease, such as preventing metastasis. Prevention of a disease does not require a total absence of cancer. For example, a decrease in tumor volume can be sufficient.

Upregulated or activation: When used in reference to the expression of a nucleic acid molecule, such as a gene, refers to any process which results in an increase in production of a gene product, such as a protein. A gene product can be RNA (such as mRNA, rRNA, tRNA, and structural RNA) or protein. Therefore, gene upregulation or activation includes processes that increase transcription of a gene or translation of mRNA.

Examples of processes that increase transcription include those that facilitate formation of a transcription initiation complex, those that increase transcription initiation rate, those that increase transcription elongation rate, those that increase processivity of transcription and those that relieve transcriptional repression (for example by blocking the binding of a transcriptional repressor). Gene upregulation can include inhibition of repression as well as stimulation of expression above an existing level. Examples of processes that increase translation include those that increase translational initiation, those that increase translational elongation and those that increase mRNA stability.

Gene upregulation includes any detectable increase in the production of a gene product. In certain examples, production of a gene product increases by at least 1.5-fold, such as at least 2-fold, at least 3-fold or at least 4-fold, as compared to a control. In one example, a control is a relative amount of gene expression in a biological sample, such as from a subject that does not have ASCVD or has not had an MI.

Additional terms commonly used in molecular genetics can be found in Benjamin Lewin, Genes V, published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

### Esophageal Adenocarcinoma (EAC) Risk

Methods are provided herein for evaluating risk, for example for determining the likelihood that a subject, such as an otherwise healthy subject, or a subject suspected or at risk of having EAC, has EAC or will likely develop EAC. In particular examples, the method can determine if a subject has or will likely develop EAC in the future. In further examples, the method can determine the likelihood that an agent is effective for treating a subject, such as a subject with EAC. In some embodiments, the method stratifies the risk of developing esophageal adenocarcinoma. In additional embodiments, the method can be used to determine the likelihood that EAC will metastasize. The method can be used for risk stratification. In yet other embodiments, the subject has been determined to be at risk for EAC based on risk factors, such as, but not limited to, clinical risk factors such as, but not limited to, GERD, Barrett's esophagus, smoking and/or alcohol use. The methods can also be used in combination with esophageal endoscopy.

In some examples, a biological sample obtained from the subject, such as, but not limited to, serum, blood, plasma, purified cells, a biopsy or tissue sample, such as a sample including esophageal tissue obtained from the subject are used to predict the subject's risk of EAC and/or developing advanced disease.

In some embodiments, the subject is apparently healthy, such as a subject who does not exhibit symptoms of EAC (for example, does not have EAC, and/or has not previously had gastroesphogeal reflux disease (GERD) or Barrett's espophagus). In some examples, a healthy subject is one that if examined by a medical professional, would be characterized as healthy and free of symptoms, such as GERD. The methods disclosed herein can be used to screen subjects for future evaluation or treatment for EAC.

In other embodiments, the methods determine the likelihood that a subject will develop EAC. In specific non-liming examples, the subject is suspected of having EAC, or is suspected of being at risk of developing EAC in the future. For example, such a subject may have GERD and/or Barrett's esophagus or use acid reducing drugs such as proton pump inhibitors or histamine antagonists to suppress gastroesophageal discomfort. The subject may be at increased risk due to smoking and/or alcohol use. The subject can have low grade dysplasia or high grade dysplasia. The methods disclosed herein can be used to determine if a subject has, or is likely to develop, EAC, and/or confirm a prior clinical suspicion of disease. The method can be used to stratify the risk of developing EAC. An increase in the level of biglycan, myeloperoxidase, protein S100-A9, and optionally annexin-A6 in a biological sample from the subject, as compared to a control, indicates that the subject is likely to develop EAC, or has EAC. In a non-limiting example, an increase in the level of biglycan, myeloperoxidase, protein S100-A9, and annexin-A6 in a biological sample from the subject, as compared to a control, indicates that the subject is likely to develop EAC, or has EAC

The detection of the expression of the markers disclosed herein can be used to assess the necessity or efficacy of an agent or therapeutic protocol for the treatment or prevention of EAC. In a non-limiting example, a decrease in the level of biglycan, myeloperoxidase, protein S100-A9, and annexin-A6 in a biological sample from the subject, as compared to a control, indicates that an agent is effective for treatment and/or prevention of EAC. In some embodiments, methods are provided for evaluating the necessity or efficacy of a treatment protocol that includes any agent designed to reverse, slow the progression of, or prevent EAC, including but not limited to treatment with surgery, radiation, a pharmaceutical agent (such as a proton pump inhibitor) or chemotherapy. In additional embodiments, the agent is an antibody, radiation, a chemotherapy, laser therapy or electrocoagulation. The antibody can specifically bind epithelial growth factor receptor, HER2, or vascular endothelial growth factor. In specific non-limiting examples, the antibody is cetuximab, trastuzumab, or bevacizumab. The agent can be surgical treatment, such as endoscopic submucosal surgical dissection (ESD), minimally invasive esophagectomy (MIE) surgery, endoscopic mucosal resection (EMR), cryoablation, esophagectomy, or radiofequency ablation (RFA).

In certain embodiments, a sample can be taken from a subject with EAC prior to initiation of therapy. After therapy is initiated, an additional sample is taken from the subject. A decrease in the amount of the markers, such as of biglycan, myeloperoxidase, protein S100-A9, and optionally annexin-A6, indicates that the therapy is efficacious. In a specific non-limiting example, a decrease in the amount of biglycan, myeloperoxidase, protein S100-A9, and annexin-A6, indicates that the therapy is efficacious.

In some embodiments, the subject can be monitored over time to evaluate the continued effectiveness of the therapeutic protocol. The effect of different dosages of an anti-EAC agent can also be evaluated, by comparing the expression of biglycan, myeloperoxidase, protein S100-A9, and optionally annexin-A6 in a sample from the subject receiving a first dose to the expression of biglycan, myeloperoxidase, protein S100-A9, and optionally annexin-A6 in a sample from the subject receiving a second (different) dose of the anti-EAC agent. In a specific non-limiting example, a decrease in the expression of biglycan, myeloperoxidase, protein S100-A9, and annexin-A6 indicates that a dose of an agent is effective for treating a subject.

In additional embodiments, a sample can be taken from a subject with high grade dysplasia or low grade dysplasia of the esophagus prior to initiation of therapy. After therapy is initiated, an additional sample is taken from the subject. A decrease in the level of biglycan, myeloperoxidase, protein S100-A9, and optionally annexin-A6, indicates that the therapy is efficacious in slowing progression and/or preventing the development of EAC. In a specific non-limiting example, a decrease in the level of biglycan, myeloperoxidase, protein S100-A9, and annexin-A6, indicates that the therapy is efficacious in slowing progression and/or preventing the development of EAC.

In addition, the subject can be monitored over time to evaluate the continued effectiveness of the therapeutic protocol. The effect of different dosages can also be evaluated, by comparing the expression of biglycan, myeloperoxidase, protein S100-A9, and optionally annexin-A6 in a sample from the subject receiving a first dose to the expression of biglycan, myeloperoxidase, protein S100-A9, and optionally annexin-A6 in a sample from the subject receiving a second (different) dose. All of biglycan, myeloperoxidase, protein S100-A9, and annexin-A6 can be measured.

The methods can be repeated 1, 2, 3, 4, 5, 6, 7, 8, 9, 10 or more times to determine the lowest dose of an agent that is effective for treating the subject, and/or the shortest duration of administration that is effective for treating the subject. In specific, non-limiting examples, the agent can be an antibody, radiation, or chemotherapy. The methods can also be used over the course of a therapeutic protocol to monitor the efficacy of the therapeutic protocol for the treatment of the subject. In specific non-limiting examples, the level of biglycan protein, myeloperoxidase protein, protein S100-A9 protein, and optionally annexin-A6 protein in a biological sample is assessed. In additional specific non-limiting examples, the level of biglycan protein, myeloperoxidase protein, protein S100-A9 protein, and annexin-A6 protein in a biological sample is assessed.

In further embodiments, methods of performing esophageal endoscopy, including but not limited to EGD, are disclosed. These methods include detecting a level of biglycan, myeloperoxidase, and protein S100-A9 in a biological sample from the subject; and comparing the level of biglycan, myeloperoxidase, and protein S100-A9 to a respective control level of biglycan, myeloperoxidase, and protein S100-A9. Esophageal endoscopy is performed on the subject if there is an increase in the level of biglycan, myeloperoxidase, and protein S100-A9 as compared to the respective control in the biological sample. The methods can also include performing an assay that detects a level of annexin-A6 in a biological sample from the subject; and comparing the level of annexin-A6 to a respective control level of annexin-A6. Esophageal endoscopy is performed on the subject if there is an increase in the level of annexin-A6 as compared to the respective control level in the biological sample. Thus, in specific non-limiting examples, methods of performing esophageal endoscopy are disclosed. These methods include detecting a level of biglycan, annexin-A6, myeloperoxidase, and protein S100-A9 in a biological sample from the subject; and comparing the level of biglycan, annexin-A6, myeloperoxidase, and protein S100-A9 to a respective control level of biglycan, annexin-A6, myeloperoxidase, and protein S100-A9. Esophageal endoscopy is performed on the subject if there is an increase in the level of biglycan, annexin-A6, myeloperoxidase, and protein S100-A9 as compared to the respective control in the biological sample.

In some embodiments, any of the methods disclosed herein can include evaluating the level of one or more of the following:

TABLE A
Markers of EAC

| HGNC Gene | UniProt/Swiss-Prot Full | GENBANK |
|---|---|---|
| SYMBOL | Name of Protein | Accession Nos. |
| BGN | biglycan | NM_001711.4 and |
| | | P21810 |
| ANXA6 | Annexin-A6 | NM 001155 and |
| | | P08133 |
| MPO | myeloperoxidase | NM_000250.1 and |
| | | P05164-1 |
| S100A9 | Protein S100-A9 | NM_002965.3 and |
| | | P06702 |

(All GENBANK ® Accession nucleic acid and amino acid sequences Incorporated by Reference as available on Dec. 1, 2013.)

Thus, the methods can include detecting the level of expression of 1, 2, 3, or all 4 of the markers listed in Table A in any combination. In some embodiments, the method includes detecting expression of biglycan, myeloperoxidase, and protein S100-A9. In additional embodiments, the method includes detecting expression of biglycan, myeloperoxidase, protein S100-A9 and annexin-A6. In yet other embodiments, the level of other markers can be assessed, such as, but not limited to, 1, 2, 3, 4, 5, 6, or all 7 of the markers listed in Table 1 below. In additional embodiments the level of resistin protein and/or mRNA can be detected.

In some embodiments, the level of biglycan protein, myeloperoxidase protein, protein S100-A9, and optionally annexin-A6 polypeptide is detected in a biological sample, such as, but not limited to, a blood, serum, or plasma sample, or a sample comprising esophageal cells, such as a biopsy sample. In other embodiments, the level of biglycan mRNA, myeloperoxidase mRNA, protein S100-A9 mRNA, and optionally annexin-A6 mRNA is detected in a biological sample, such as, but not limited to, a sample comprising esophageal cells, such as a biopsy sample. Proteins and mRNA can be evaluated. Thus, in specific non-limiting examples, the method includes detecting expression of biglycan polypeptide, myeloperoxidase polypeptide, and protein S100-A9 polypeptide, and optionally detecting expression of annextin-A6 polypeptide. In other specific non-limiting examples, the method includes detecting expression of biglycan mRNA, myeloperoxidase mRNA, and protein S100-A9 mRNA, and optionally detecting expression of annextin-A6 mRNA.

In some embodiments, methods are provided for detecting or determining the likelihood that a subject will develop EAC. The methods can include performing one or more assays that detects expression of biglycan, myeloperoxidase, and protein S100-A9 in a biological sample from the subject, and comparing the level of expression of biglycan, myeloperoxidase, and protein S100-A9 to a respective control level of biglycan, myeloperoxidase, and protein S100-A9. An increase in expression level of biglycan, myeloperoxidase, and protein S100-A9 as compared to the respective control level indicates that the subject has or will develop EAC. In some embodiments, the method also includes performing an assay that detects expression of annexin-A6 in a biological sample from the subject; and comparing the level of expression of annexin-A6 to a respective control level of annexin-A6. The detection of an increase in expression of annexin-A6 compared to the respective control level of annexin-A6, in conjunction with the increase in expression of biglycan, myeloperoxidase and protein S100-A9, indicates that the subject has or will develop EAC. Thus, in some embodiments, the methods include performing one or more assays that detects expression of biglycan, annexin-A6, myeloperoxidase, and protein S100-A9 in a biological sample from the subject, and comparing the level of expression of biglycan, annexin-A6, myeloperoxidase, and protein S100-A9 to a respective control level of biglycan, annexin-A6, myeloperoxidase, and protein S100-A9.

The control can be a standard value of biglycan, myeloperoxidase, and protein S100-A9, and annexin-A6 respectively, or can be the level of biglycan, myeloperoxidase, and protein S100-A9, and annexin-A6, respectively, in one or more subjects known not to have EAC. Proteins and/or mRNA can be evaluated. In one specific non-limiting example, the level of biglycan protein, myeloperoxidase protein, and protein S100-A9, and optionally annexin-A6 protein is measured in a biological sample from the subject, such as a tissue, blood, serum or plasma sample.

Methods are also provided for determining if a pharmaceutical agent is effective for treatment or prevention of EAC in a subject. In specific non-liming examples, the subject can have EAC. In other embodiments, the subject has GERD, Barrett's esophagus, or another condition associated with risk of EAC, such as low grade dysplasia or high grade dysplasia.

In some embodiments, the methods include performing one or more assays that detect expression of biglycan, myeloperoxidase, and protein S100-A9 in a biological sample from the subject administered the agent; and comparing the level of expression of biglycan, myeloperoxidase, and protein S100-A9 to a respective control level of biglycan, myeloperoxidase, and protein S100-A9. The detection of a decrease in expression of biglycan, myeloperoxidase, and protein S100-A9 as compared to the respective control indicates that the agent is effective for the treatment or prevention of EAC in the subject. In additional embodiments, the method includes performing an assay that detects expression of annexin-A6 in a biological sample from the subject; and comparing the level of expression of annexin-A6 to a respective control level of annexin-A6. The detection of a decrease in expression of annexin-A6 as compared to the respective control level of annexin-A6 indicates that the pharmaceutical agent is effective for the treatment or prevention of EAC in the subject. In additional embodiments, the methods include assessing all of the biglycan, myeloperoxidase, protein S100-A9, and annexin-A6.

In any of the methods disclosed herein, proteins and/or mRNA can be evaluated. In one specific non-limiting example, the level of biglycan, myeloperoxidase, and protein S100-A9, and optionally annexin-A6 protein is measured in a biological sample from the subject, such as a tissue, blood, serum or plasma sample.

### Methods for Detection of Proteins

In some examples, the level of one or more proteins is analyzed by detecting and quantifying the protein in a biological sample. In particular examples, one or more proteins corresponding to the markers listed in Table A are analyzed. Suitable biological samples include samples containing protein, such as blood, serum, plasma, urine, saliva, cells, for example peripheral blood mononuclear cells, B cells, T cells and/or monocytes, and tissue samples, such as esophageal biopsy samples. Detecting an alteration in the amount of one or more of biglycan, myeloperoxidase, and protein S100-A9, and optionally annexin-A6, using the methods disclosed herein indicates the prognosis or diagnosis of the subject, or indicates if a therapy is effective for treating a subject.

Expression of proteins is the level of protein in a biological sample. Expression includes, but is not limited to, the production of the protein by translation of an mRNA and the half-life of the protein.

Any standard immunoassay format (such as ELISA, Western blot, or RIA assay) can be used to measure protein levels. Immunohistochemical techniques can be utilized. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998), and Harlow & Lane, Antibodies, A Laboratory Manual, Cold Spring Harbor Publications, New York (1988); these references disclose a number of immunoassay formats and conditions that can be used to determine specific immunoreactivity. Generally, immunoassays include the use of one or more specific binding agents (such as antibodies) that specifically recognizes and can bind a molecule of interest, such biglycan, myeloperoxidase, and protein S100-A9, and annexin-A6. Such binding agents can include a detectable label (such as a radiolabel, fluorophore or enzyme), that permits detection of the binding to the protein and determination of relative or absolute quantities of the molecule of interest in the sample. Although the details of the immunoassays may vary with the particular format employed, the method of detecting the protein in a sample generally includes the steps of contacting the sample with an antibody, which specifically binds to the protein under immunologically reactive conditions to form an immune complex between the antibody and the protein, and detecting the presence of and/or quantity of the immune complex (bound antibody), either directly or indirectly. The antibody can be a polyclonal or monoclonal antibody, or fragment thereof. In some examples, the antibody is a humanized antibody. In additional examples, the antibody is a chimeric antibody.

The antibodies can be labeled. Suitable detectable markers are described and known to the skilled artisan. For example, various enzymes, prosthetic groups, fluorescent materials, luminescent materials, magnetic agents, and radioactive materials can be used. Non-limiting examples of suitable enzymes include horseradish peroxidase, alkaline phosphatase, beta-galactosidase, or acetylcholinesterase. Non-limiting examples of suitable prosthetic group complexes include streptavidin/biotinm and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary magnetic agent is gadolinium, and non-limiting exemplary radioactive labels include ¹²⁵I, ¹³¹I, ³⁵S or ³H. Additional examples are disclosed above.

In another embodiment, the antibody that binds the protein of interest (the first antibody) is unlabeled and a second antibody or other molecule that can bind the antibody that binds the protein of interest is utilized. As is well known to one of skill in the art, a second antibody is chosen that is able to specifically bind the specific species and class of the first antibody. For example, if the first antibody is a mouse IgG, then the secondary antibody may be a goat anti-mouse-IgG. Other molecules that can bind to antibodies include, without limitation, Protein A and Protein G, both of which are available commercially.

Quantitation of proteins can be achieved by immunoassay. The amount of proteins can be assessed and optionally in a control sample. The amounts of protein in the sample from the subject of interest can be compared to levels of the protein found in samples form control subjects or to another control (such as a standard value or reference value). A significant increase or decrease in the amount can be evaluated using statistical methods known in the art.

In some non-limiting examples, a sandwich ELISA can be used to detect the presence or determine the amount of a protein in a sample. In this method, a solid surface is first coated with an antibody that specifically binds the protein of interest. The test sample containing the protein (such as, but not limited to, a blood, plasma, serum, or urine sample), is then added and the antigen is allowed to react with the bound antibody. Any unbound antigen is washed away. A known amount of enzyme-labeled protein—specific antibody is then allowed to react with the bound protein. Any excess unbound enzyme-linked antibody is washed away after the reaction. The substrate for the enzyme used in the assay is then added and the reaction between the substrate and the enzyme produces a color change. The amount of visual color change is a direct measurement of specific enzyme-conjugated bound antibody, and consequently the quantity of the protein present in the sample tested.

In an alternative example, a protein can be assayed in a biological sample by a competition immunoassay utilizing protein standards labeled with a detectable substance and an unlabeled antibody that specifically binds the protein of interest. In this assay, the biological sample (such as, but not limited to, a blood, plasma, serum, or urine sample), the labeled protein standards and the antibody that specifically binds the protein of interest are combined and the amount of labeled protein standard bound to the unlabeled antibody is determined. The amount of protein in the biological sample is inversely proportional to the amount of labeled protein standard bound to the antibody that specifically binds the protein of interest.

Mass spectrometry is particularly suited to the identification of proteins from biological samples, such biglycan, myeloperoxidase, and protein S100-A9, and annexin-A6. Mass spectrometry also is particularly useful in the quantitation of peptides in a biological sample, for example using isotopically labeled peptide standards. The application of mass spectrometric techniques to identify proteins in biological samples is known in the art and is described, for example, in Akhilesh et al., Nature, 405:837-846, 2000; Dutt et al., Curr. Opin. Biotechnol., 11:176-179, 2000; Gygi et al., Curr. Opin. Chem. Biol., 4 (5): 489-94, 2000; Gygi et al., Anal. Chem., 72 (6): 1112-8, 2000; and Anderson et al., Curr. Opin. Biotechnol., 11:408-412, 2000.

Separation of ions according to their m/z ratio can be accomplished with any type of mass analyzer, including quadrupole mass analyzers (Q), time-of-flight (TOF) mass analyzers (for example, linear or reflecting) analyzers, magnetic sector mass analyzers, 3D and linear ion traps (IT), Fourier-transform ion cyclotron resonance (FT-ICR) analyzers, Orbitrap analyzers (like LTQ-Orbitrap LC/MS/MS), and combinations thereof (for example, a quadrupole-time-of-flight analyzer, or Q-TOF analyzer). A triple quadropole instrument can be used such as the Q-trap.

In some embodiments, the mass spectrometric technique is tandem mass spectrometry (MS/MS). Typically, in tandem mass spectrometry a protein gene product, such as biglycan, myeloperoxidase, protein S100-A9, and annexin-A6, entering the tandem mass spectrometer is selected and subjected to collision induced dissociation (CID). The spectrum of the resulting fragment ion is recorded in the second stage of the mass spectrometry, as a so-called CID or ETD spectrum. Because the CID or ETD process usually causes fragmentation at peptide bonds and different amino acids for the most part yield peaks of different masses, a CID or ETD spectrum alone often provides enough information to determine the presence of biglycan, myeloperoxidase, protein S100-A9, and annexin-A6. Suitable mass spectrometer systems for MS/MS include an ion fragmentor and one, two, or more mass spectrometers, such as those described above. Examples of suitable ion fragmentors include, but are not limited to, collision cells (in which ions are fragmented by causing them to collide with neutral gas molecules), photo dissociation cells (in which ions are fragmented by irradiating them with a beam of photons), and surface dissociation fragmentor (in which ions are fragmented by colliding them with a solid or a liquid surface). Suitable mass spectrometer systems can also include ion reflectors.

Prior to mass spectrometry, the sample can be subjected to one or more dimensions of chromatographic separation, for example, one or more dimensions of liquid or size exclusion chromatography. Representative examples of chromatographic separation include paper chromatography, thin layer chromatography (TLC), liquid chromatography, column chromatography, high performance liquid chromatography (HPLC), fast protein liquid chromatography (FPLC), ion exchange chromatography, size exclusion chromatography, affinity chromatography, high performance liquid chromatography (HPLC), nano-reverse phase liquid chromatography (nano-RPLC), polyacrylamide gel electrophoresis (PAGE), capillary electrophoresis (CE), reverse phase high performance liquid chromatography (RP-HPLC) or other suitable chromatographic techniques. Thus, in some embodiments, the mass spectrometric technique is directly or indirectly coupled with a one, two or three dimensional liquid chromatography technique, such as column chromatography, high performance liquid chromatography (HPLC or FPLC), reversed phase, ion exchange chromatography, size exclusion chromatography, affinity chromatography (such as protein or peptide affinity chromatography, immunoaffinity chromatography, lectin affinity chromatography, etc.), or one, two or three dimensional polyacrylamide gel electrophoresis (PAGE), or one or two dimensional capillary electrophoresis (CE) to further resolve the biological sample prior to mass spectrometric analysis.

A variety of mass spectrometry methods, including iTRAQ® and MRM, can be used. In some embodiments, quantitative spectroscopic methods, such as SELDI, are used to analyze protein expression in a sample. In one example, surface-enhanced laser desorption-ionization time-of-flight (SELDI-TOF) mass spectrometry is used to detect protein expression, for example by using the PROTEINCHIP™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption. Additional methods are disclosed in the examples section below.

Briefly, one version of SELDI uses a chromatographic surface with a chemistry that selectively captures analytes of interest, such as one or more proteins of interest. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector.

In an additional example, the method may include detection of a protein of interest in a sample using an electrochemical immunoassay method. See, e.g., Yu et al., J. Am. Chem. Soc., 128:11199-11205, 2006; Mani et al., ACS Nano, 3:585-594, 2009; Malhotra et al., Anal. Chem., 82:3118-3123, 2010. In this method, an antibody that specifically binds the protein of interest is conjugated to terminally carboxylated single-wall carbon nanotubes (SWNT), multi-wall carbon nanotubes (MWCNT), or gold nanoparticles (AuNP), which are attached to a conductive surface. A sample (such as a blood, plasma or serum sample) is contacted with the SWNTs, MWCNTs, or AuNPs, and protein in the sample binds to the primary antibody. A second antibody conjugated directly or indirectly to a redox enzyme (such as horseradish peroxidase (HRP), cytochrome c, myoglobin, or glucose oxidase) binds to the primary antibody or to the protein (for example, in a “sandwich” assay). In some examples, the second antibody is conjugated to the enzyme. In other examples, the second antibody and the enzyme are both conjugated to a support (such as a magnetic bead). Signals are generated by adding enzyme substrate (e.g. hydrogen peroxide if the enzyme is HRP) to the solution bathing the sensor and measuring the current produced by the catalytic reduction.

In a particular example, the method includes a first antibody that specifically binds the protein of interest attached to an AuNP sensor surface. A sample (such as, but not limited to, a blood, plasma, serum, or urine sample) is contacted with the AuNP sensor including the first antibody. After the protein of interest binds to the first (capture) antibody (Ab1) on the electrode, a horseradish peroxidase (HRP)-labeled second antibody that specifically binds the protein of interest (HRP-Ab2) or beads conjugated to both a second antibody that binds the protein of interest and HRP are incubated with the sensor, allowing the second antibody to bind to the protein of interest. Biocatalytic electrochemical reduction produces a signal via reduction of peroxide activated enzyme following addition of hydrogen peroxide. Use of HRP is advantageous for arrays since immobilization of the electroactive enzyme label on the electrode eliminates electrochemical crosstalk between array elements, which can occur when detecting soluble electroactive product.

In some embodiments, isobaric tags for relative and absolute quantification (iTRAQ®) reagents are utilized to enable simultaneous quantification of multiple samples. The iTRAQ technology utilizes isobaric tags to label the primary amines of peptides and proteins. Multiple samples can be run simultaneously using different iTRAQ® reagents that label the individual samples with different mass identifiers. By way of example, sample one can be labeled with a mass identifier (or mass tag) that has a molecular weight of 114 amu, while sample two mass identifier (or mass tag) can have a molecular weight of 117. When the samples are combined and subjected to mass spectrometric analysis, the reporter ion in the tandem mass spectra of a peptide from sample two will have a predictable mass difference of three amu, compared to the reporter ion from sample one. This relative intensities of different reporter ions can be used for relative quantification of a peptide (and hence the protein from which they were derived).

In multiple reaction monitoring (MRM), tryptic peptides are used as markers for the abundance of specific proteins of interest, such as biglycan, myeloperoxidase, protein S100-A9, and optionally annexin-A6. This selection is relatively straightforward if the protein has been identified by MS, such that the peptides are observable in a mass spectrometer (for example an LTQ Orbitrap). The process of establishing an MRM assay for a protein consists of a number of steps: 1) selection of the appropriate peptide(s) unique to the protein of interest and showing high MS signal response (prototypic peptides) which will help maximize the sensitivity of the assay; 2) selection of predominant peptide fragments specific (MS/MS) for the parent peptide (useful MRM transition); 3) for each peptide-fragment pair, optimization of specific MS parameters (for example, the collision energy) to maximize the signal response/sensitivity; 4) validation of the MRM assay to confirm peptide identity, for example by acquiring a full MS2 spectrum of the peptide in the triple quadrupole MS instrument used for MRM; 5) extraction of the final “coordinates” of the MRM assay, including the selected peptide and peptide fragments, the corresponding mass-to-charge ratios, the fragment intensity ratios, the associated collision energy, and the chromatographic elution time to be optionally used in time-constrained MRM analyses. In some examples, isotopically labeled internal peptide standards (with known concentrations determined by amino acid analysis) are used to facilitate absolute quantitation of selected peptides.

The concentration of the protein of interest, such as biglycan, myeloperoxidase, protein S100-A9, and annexin-A6, that is detected can be compared to a control, such as the concentration of the protein in a subject known not to have EAC, and/or known not to have had Barrett's esophagus, and/or known not to have GERD. In other embodiments, the control is a standard value, such as a value that represents an average concentration of the protein of interest expected in a subject who does not have EAC and/or Barrett's esophagus, and/or known not to have GERD.

### Methods for Detection of mRNA

Gene expression can be evaluated by detecting mRNA encoding the gene of interest. Thus, the disclosed methods can include evaluating mRNA encoding biglycan, myeloperoxidase, protein S100-A9, and optionally annexin-A6. Any of the methods disclosed above can utilize the detection of mRNA.

RNA can be isolated from a sample from a subject, such as an esophageal biopsy sample. RNA can also be isolated from a control, such as esophageal cells from a healthy subject, for example a subject known not to have EAC or be at risk for EAC, using methods well known to one skilled in the art, including commercially available kits. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Biotechniques 6:56-60 (1988), and De Andres et al., Biotechniques 18:42-44 (1995). In one example, RNA isolation can be performed using purification kit, buffer set and protease from commercial manufacturers, such as QIAGEN® (Valencia, Calif.), according to the manufacturer's instructions. For example, total RNA from cells in culture (such as those obtained from a subject) can be isolated using QIAGEN® RNeasy® mini-columns. Other commercially available RNA isolation kits include MASTERPURE® Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from a biological sample can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using Northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, such as from subject that is undergoing treatment, to characterize patterns of gene expression, to discriminate between closely related mRNAs, and to analyze RNA structure.

Methods for quantitating mRNA are well known in the art. In some examples, the method utilizes RT-PCR. For example, extracted RNA can be reverse-transcribed using a GENEAMP® RNA PCR kit (Perkin Elmer, Calif., USA), following the manufacturer's instructions.

For example, TAQMAN® RT-PCR can be performed using commercially available equipment. The system can include a thermocycler, laser, charge-coupled device (CCD) camera, and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by an experimental treatment. RNAs commonly used to normalize patterns of gene expression are mRNAs for the housekeeping genes GAPDH, f3-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems (Foster City, Calif.).

The steps of a representative protocol for quantitating gene expression using fixed, paraffin-embedded tissues as the RNA source, including mRNA isolation, purification, primer extension and amplification are given in various published journal articles (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tissue samples or adjacent non-diseased tissue. The RNA is then extracted, and protein and DNA are removed. Alternatively, RNA is isolated directly from a tissue sample. After analysis of the RNA concentration, RNA repair and/or amplification steps can be included, if necessary, and RNA is reverse transcribed using gene specific promoters followed by RT-PCR.

The primers used for the amplification are selected so as to amplify a unique segment of the gene of interest (such as mRNA encoding biglycan, myeloperoxidase, protein S100-A9, and optionally annexin-A6). In some embodiments, expression of other genes is also detected. Primers that can be used to amplify mRNAs of interest are commercially available or can be designed and synthesized according to well-known methods.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some examples, gene expression is identified or confirmed using the microarray technique. Thus, the expression profile can be measured in either fresh or paraffin-embedded tissue, using microarray technology. In this method, nucleic acid sequences of interest (including cDNAs and oligonucleotides) are plated, or arrayed, on a microchip substrate. The arrayed sequences are then hybridized with isolated nucleic acids (such as cDNA or mRNA) from cells or tissues of interest. Just as in the RT-PCR method, the source of mRNA typically is total RNA isolated from tissue or cells, and optionally from corresponding tissues or cells from a subject known not to be at risk for ASCVD and/or MI.

In a specific embodiment of the microarray technique, PCR amplified inserts of cDNA clones are applied to a substrate in a dense array. In some examples, the array includes probes specific to biglycan, myeloperoxidase, protein S100-A9, andoptionally annexin-A6. In some examples, probes specific for these nucleotide sequences are applied to the substrate, and the array can consist essentially of, or consist of these sequences. The microarrayed nucleic acids are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for genes of interest, such as biglycan, myeloperoxidase, protein S100-A9, and optionally annexin-A6. Microarray analysis can be performed by commercially available equipment, following manufacturer's protocols, such as are supplied with Affymetrix GENECHIP® technology (Affymetrix, Santa Clara, Calif.), or Agilent's microarray technology (Agilent Technologies, Santa Clara, Calif.).

Serial analysis of gene expression (SAGE) is another method that allows the simultaneous and quantitative analysis of a large number of gene transcripts, without the need of providing an individual hybridization probe for each transcript. First, a short sequence tag (about 10-14 base pairs) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag (see, for example, Velculescu et al., Science 270:484-7, 1995; and Velculescu et al., Cell 88:243-51, 1997).

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as biglycan, myeloperoxidase, protein S100-A9, and optionally annexin-A6. Sample cells or tissues are treated to increase their permeability to allow a probe to enter the cells. The probe is added to the treated cells, allowed to hybridize at pertinent temperature, and excess probe is washed away. A complementary probe is labeled so that the probe's location and quantity in the tissue can be determined, for example, using autoradiography, fluorescence microscopy or immunoassay. The sample may be any sample of interest.

In situ PCR is the PCR-based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, ³H-CTP or biotin-16-dUTP), which have been incorporated into the PCR products during thermal cycling.

In some embodiments of the detection methods, the expression of one or more “housekeeping” genes or “internal controls” can also be evaluated. These terms include any constitutively or globally expressed gene (or protein) whose presence enables an assessment of gene (or protein) levels. Such an assessment includes a determination of the overall constitutive level of gene transcription and a control for variations in RNA (or protein) recovery. The methods can also evaluate expression of other markers.

The concentration of the mRNA of interest, such as a mRNA corresponding to biglycan, myeloperoxidase, protein S100-A9, and optionally annexin-A6, that is detected is compared to a control, such as the concentration of the mRNA in a subject known not to have EAC, and/or known not to have Barrett's esophagus, and/or known not to have GERD. In other embodiments, the control is a standard value, such as a value that represents an average concentration of the mRNA of interest expected in a subject who does not have EAC and/or Barrett's esophagus, and/or GERD.

### Arrays

In particular embodiments provided herein, arrays can be used to evaluate gene expression. When describing an array that consists essentially of probes or primers specific for the biglycan, myeloperoxidase, protein S100-A9, and optionally annexin-A6, such an array includes probes or primers specific for these genes, and can further include control probes (for example to confirm the incubation conditions are sufficient). In some examples, the array can consist essentially of probes or primers specific for biglycan, myeloperoxidase, protein S100-A9, and optionally includes probes or primers specific for annexin-A6. The array can further include one or more control probes. In some examples, the array may further include additional, such as about 5, 10, 20, 30, 40, 50, 60, or 70 additional nucleic acids. Exemplary control probes include GAPDH, β-actin, and 18S RNA. In one example, an array is a multi-well plate (e.g., 96 or 384 well plate). The oligonucleotide probes or primers can further include one or more detectable labels, to permit detection of hybridization signals between the probe and target sequence.

1. Array Substrates

The solid support of the array can be formed from an organic polymer. Suitable materials for the solid support include, but are not limited to: polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof (see U.S. Pat. No. 5,985,567).

In general, suitable characteristics of the material that can be used to form the solid support surface include: being amenable to surface activation such that upon activation, the surface of the support is capable of covalently attaching a biomolecule such as an oligonucleotide thereto; amenability to “in situ” synthesis of biomolecules; being chemically inert such that at the areas on the support not occupied by the oligonucleotides or proteins (such as antibodies) are not amenable to non-specific binding, or when non-specific binding occurs, such materials can be readily removed from the surface without removing the oligonucleotides or proteins (such as antibodies).

In another example, a surface activated organic polymer is used as the solid support surface. One example of a surface activated organic polymer is a polypropylene material aminated via radio frequency plasma discharge. Other reactive groups can also be used, such as carboxylated, hydroxylated, thiolated, or active ester groups.

2. Array Formats

A wide variety of array formats can be employed in accordance with the present disclosure. One example includes a linear array of oligonucleotide bands, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use (see U.S. Pat. No. 5,981,185). In some examples, the array is a multi-well plate. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range. The array can include biaxially oriented polypropylene (BOPP) films, which in addition to their durability, exhibit low background fluorescence.

The array formats of the present disclosure can be included in a variety of different types of formats. A “format” includes any format to which the solid support can be affixed, such as microtiter plates (e.g., multi-well plates), test tubes, inorganic sheets, dipsticks, and the like. For example, when the solid support is a polypropylene thread, one or more polypropylene threads can be affixed to a plastic dipstick-type device; polypropylene membranes can be affixed to glass slides. The particular format is, in and of itself, unimportant. All that is necessary is that the solid support can be affixed thereto without affecting the functional behavior of the solid support or any biopolymer absorbed thereon, and that the format (such as the dipstick or slide) is stable to any materials into which the device is introduced (such as clinical samples and hybridization solutions).

The arrays of the present disclosure can be prepared by a variety of approaches. In one example, oligonucleotide or protein sequences are synthesized separately and then attached to a solid support (see U.S. Pat. No. 6,013,789). In another example, sequences are synthesized directly onto the support to provide the desired array (see U.S. Pat. No. 5,554,501). Suitable methods for covalently coupling oligonucleotides and proteins to a solid support and for directly synthesizing the oligonucleotides or proteins onto the support are known to those working in the field; a summary of suitable methods can be found in Matson et al., Anal. Biochem. 217:306-10, 1994. In one example, the oligonucleotides are synthesized onto the support using conventional chemical techniques for preparing oligonucleotides on solid supports (such as PCT applications WO 85/01051 and WO 89/10977, or U.S. Pat. No. 5,554,501).

A suitable array can be produced using automated means to synthesize oligonucleotides in the cells of the array by laying down the precursors for the four bases in a predetermined pattern. Briefly, a multiple-channel automated chemical delivery system is employed to create oligonucleotide probe populations in parallel rows (corresponding in number to the number of channels in the delivery system) across the substrate. Following completion of oligonucleotide synthesis in a first direction, the substrate can then be rotated by 90° to permit synthesis to proceed within a second set of rows that are now perpendicular to the first set. This process creates a multiple-channel array whose intersection generates a plurality of discrete cells.

The oligonucleotides can be bound to the polypropylene support by either the 3′ end of the oligonucleotide or by the 5′ end of the oligonucleotide. In one example, the oligonucleotides are bound to the solid support by the 3′ end. However, one of skill in the art can determine whether the use of the 3′ end or the 5′ end of the oligonucleotide is suitable for bonding to the solid support. In general, the internal complementarity of an oligonucleotide probe in the region of the 3′ end and the 5′ end determines binding to the support.

In particular examples, the oligonucleotide probes on the array include one or more labels, that permit detection of oligonucleotide probe:target sequence hybridization complexes.

### Kits

Kits are also provided. The kit can include probes, primers, or antibodies specific for biglycan, myeloperoxidase, and protein S100-A9, and can further include control probes, primers, and antibodies (for example to confirm the incubation conditions are sufficient). In some examples, the kit includes probes, primers and/or antibodies specific for biglycan, myeloperoxidase, protein S100-A9, and optionally probes, primers and/or antibodies specific for annexin-A6. The kit can further include one or more control probes, primers and/or antibodies

The kit can include a container and a label or package insert on or associated with the container. Suitable containers include, for example, bottles, vials, syringes, etc. The containers may be formed from a variety of materials such as glass or plastic. The container typically holds a composition including one or more of the probes, primers and/or antibodies. In several embodiments the container may have a sterile access port.

A label or package insert indicates that the composition is of use for evaluating if a subject is at risk for EAC, or if a therapeutic agent is of use of the treatment of a subject. The label or package insert typically will further include instructions for use, such as particular assay conditions. The package insert typically includes instructions customarily included in commercial packages of products that contain information about the indications, usage, contraindications and/or warnings concerning the use of such products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

The following examples are provided to illustrate certain particular features and/or embodiments. These examples should not be construed to limit the disclosure to the particular features or embodiments described.

### EXAMPLES

Esophageal Adenocarcinoma (EAC) is associated with a dismal prognosis, with a five-year survival of less than 15%. The identification of cancer biomarkers advances the possibility for early detection and better monitoring of tumor progression and/or response to therapy. The study disclosed below presents results of the development of a serum based assay for-proteins (biglycan, myeloperoxidase (MPO), annexin-A6 (ANXA6), and protein S100-A9; B-MAP ©) for EAC that are clinically relevant and followed a distinct pattern of expression along the sequence of disease progression.

A vertically integrated proteomics-based biomarker approach was used to identify serum biomarkers for detection of EAC. Liquid chromatography-mass spectrometry (LC-MS/MS) analysis was performed on FFPE tissue samples that were collected from across the Barrett's esophagus (BE)-EAC disease spectrum. The MS-based spectral count data was used for the selection of candidate serum biomarkers. The serum ELISA data was validated in an independent cohort and used to develop a multi-parametric risk assessment model to predict the presence of disease.

With a minimum threshold of 10 spectral counts, 351 proteins were identified as differentially abundant along the spectrum of BE, HGD and EAC (p<0.05). Eleven proteins from this dataset were then tested using ELISAs in serum samples of which five proteins were significantly elevated in abundance in the EAC patients compared to normal controls, which mirrored trends across the disease spectrum present in the tissue data. Using serum data, a Bayesian Rule Learning predictive model with four biomarkers was developed to accurately classify disease class; the cross-validation results for the merged dataset yielded accuracy of 87% and AUROC of 93%. These biomarkers and/or subsets thereof can be used for detection of EAC and selection of subjects for further diagnostic assays and/or treatment.

### Example 1

### Materials and Methods

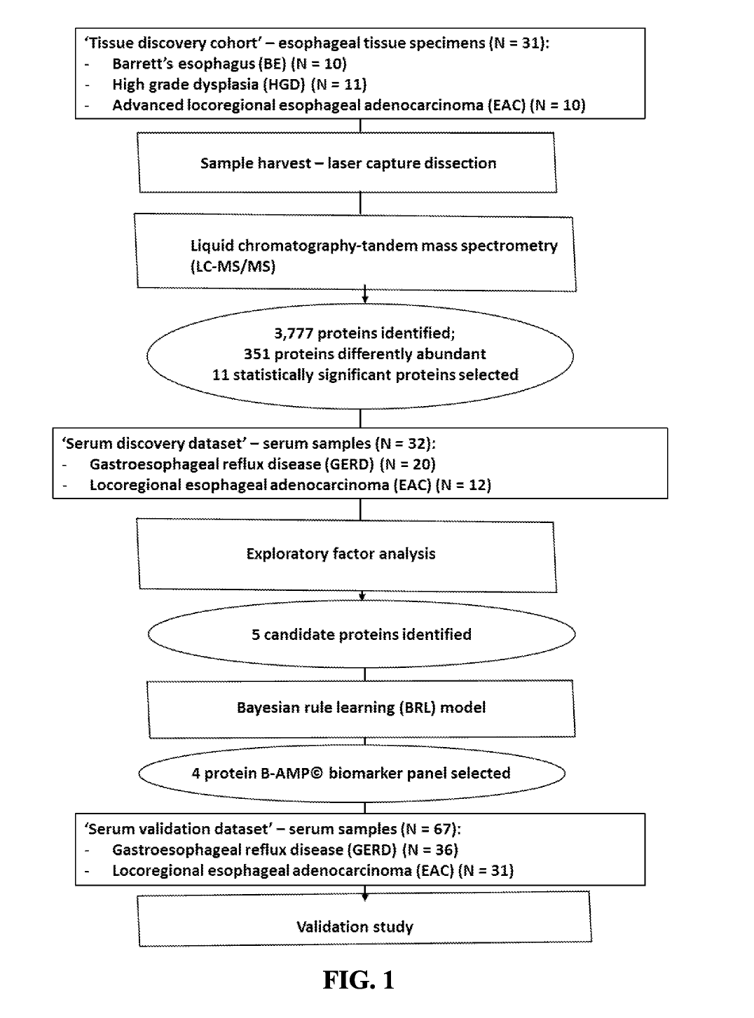

FIG. 1 outlines the overall study schema with the patient populations and methods used.

Proteomic Biomarker Identification from Barrett's Esophagus (BE), High-Grade Dysplasia (HGD) and Esophageal Adenocarcinoma (EAC) Tissues:

To identify candidate protein biomarkers associated with disease progression, a mass spectrometry based discovery study was first performed using appropriate pathologically-defined esophageal tissue specimens. The tissue discovery data was generated from archival de-identified Formalin-Fixed, Paraffin-Embedded (FFPE) blocks. The cohort consisted of 10 BE, 10 HGD and 9 locoregional EAC unpaired patient samples (‘Tissue discovery cohort’—FIG. 1).

A single-tube experimental protocol was used to digest proteins from FFPE tissue sections with trypsin and the resultant tryptic peptides were analyzed by liquid chromatography-tandem mass spectrometry (LC-MS/MS) for an exploratory proteomic analysis of BE, HGD and EAC. Approximately 40,000 cells per sample were collected by laser capture micro dissection (LCM); the tryptic digests were analyzed in duplicate by nanoflow LC-MS/MS using a hybrid linear ion trap-ORBITRAP® mass spectrometer (MS). The primary tandem MS/MS data were searched with SEQUEST® against the human proteome database for peptide identification and against a “decoy” human proteome database where the protein sequences are reversed to maintain a false discovery rate of less than 1% (Elias and Gygi, Nat. Methods 2007; 4(3) 207-14). The resulting peptide lists were integrated using a suite of in-house MATLAB® enabled relational database tools yielding spectral counts for identified proteins.

A quantitative estimate of the relative abundance of the identified proteins from these data sets was obtained by comparison of their spectral count values between BE-, HGD-, and EAC-derived cells. To determine statistically significant differentially abundant proteins from each tissue type, a Kruskal-Wallis non-parametric analysis of variance test was applied (Kruskal-Wallis test is a one-way analysis of variance by ranks and determines statistically significant differences between two or more groups of an independent variable on a continuous or ordinal dependent variable); proteins with significant differences were utilized for further hierarchical clustering analysis.

Digestion of Laser Microdissected PPFE Tissues:

Heat-induced trypsin digestion was applied to the LCM cells to extract peptides as described (Teng et al., J Proteome Res 2010; 9(8): 4161-9). Samples were resuspended in 100 μl of 100 mM NH₄HCO₃/20% acetonitrile and then heated at 90° C. for 1 hour, followed by 65° C. for 2 hours. Trypsin digestion was carried out by adding 500 ng sequencing grade modified trypsin (Promega, Madison, Wis.) followed by overnight incubation at 37° C. After a rapid spin, the aqueous solution was transferred to a new Eppendorf tube, lyophilized and then resuspended in 100 μl 0.1% trifluoroacetic acid (TFA), followed by desalting with PepClean C-18 Spin Columns (PIERCE, Rockford, Ill.), vacuum-dried and resuspended in 25 μl 0.1% TFA. The BCA assay (PIERCE, Rockford, Ill.) was used to determine peptide concentration.

Liquid Chromatography Tandem Mass Spectrometry (LC-MS/MS) Analysis of Peptides:

The tryptic digests were analyzed in duplicate (1 μg for each injection) by reverse-phase LC-MS/MS using a nanoflow LC (Dionex Ultimate 3000, Dionex Corporation, Sunnyvale, Calif.) coupled online to an LTQ/ORBITRAP® XL hybrid mass spectrometer (ThermoFisher Scientific Inc., San Jose, Calif.). Peptide separation was performed using 75 μm inner diameter×360 μm outer diameter×20 cm long fused silica capillary columns (Polymicro Technologies, Phoenix, Ariz.) slurry packed in house with 5 μm, 300 Å pore size C-18 silica-bonded stationary phase (Jupiter, Phenomenex, Torrance, Calif.). Following sample injection onto a C-18 trap column (Dionex), the column was washed for 3 min with mobile phase A (2% acetonitrile, 0.1% formic acid) at a flow rate of 30 μl/min. Peptides were eluted using a linear gradient of 0.30% mobile phase B (0.1% formic acid in acetonitrile)/minute for 130 min, then to 95% B in an additional 10 min, all at a constant flow rate of 250 nl/min. Column washing was performed at 95% B for 20 min, after which the column was re-equilibrated in mobile phase A prior to subsequent injections. The LTQ/Orbitrap XL MS was configured to collect high resolution (R=60,000 at m/z 400) broadband mass spectra (m/z 375-1800) from which the 7 most abundant peptide molecular ions dynamically determined from the MS scan were selected for MS/MS using a 30% normalized collision-induced dissociation energy. Dynamic exclusion was utilized to minimize redundant selection of peptides for MS/MS analysis.

MS and MS/MS Data Analysis:

For relative quantitation using spectral count, tandem mass spectra were searched against the UniProt human proteome database (June/2009 release) from the European Bioinformatics Institute (http://www.ebi.ac.uk/integr8) using SEQUEST® (ThermoFisher Scientific Inc.) with variable modification of methionine (oxidation, +15.9949 Da). The mass tolerance for the precursor ions and fragment ions were set to 20 ppm and 1 Da respectively. Peptides were considered legitimately identified if they achieved specific charge state and proteolytic cleavage-dependent cross-correlation (Xcorr) scores of 1.9 for [M+H]¹⁺, 2.2 for [M+2H]²⁺, and 3.5 for [M+3H]³⁺, and a minimum delta correlation score (ΔCn) of 0.08. An in-house MATLAB® script was used to combine the total number of CID spectra that resulted in positive identification of any peptides for a given protein (spectral count).

Patient Study Populations and Serum Sample Collection:

For initial evaluation of the abundances of the candidate protein biomarkers in serum, a ‘serum discovery dataset’ of samples was collected from a total of 32 patients: 20 with a clinical diagnosis of gastroesophageal reflux disease (GERD) and 12 with advanced loco-regional EAC (T2N1 to T3N0).

For this purpose, venous blood samples (4 ml) from normal control GERD and EAC patients were drawn using standard venipuncture into red/yellow top VACUETTE® Serum Clot Activator with Gel Separator Blood Collection Tubes (Greiner-Bio-One #454067, Monroe, N.C., USA) and placed upright 30 to 60 minutes until clot formation. The tubes were centrifuged in a swinging bucket rotor (1,300 g×20 min) and the serum was pipetted and distributed as 200 aliquots in 1.5 ml cryovials for storage at −80° C. To ensure consistency and reliability in the subsequent analyses, no more than one freeze-thaw cycle was allowed for any sample.

Enzyme Linked Immunosorbent Assay (ELISA) Testing for the Candidate Serum Protein Biomarker Panel:

Commercial ELISA kits were used to quantitate the abundance of the candidate biomarkers in serum.

The following ELISA kits were used: alpha-1-antitrypsin (A1A), myeloperoxidase (MPO), apolipoprotein A-I (APO A1) (Immunology Consultants Laboratory, Inc., #E-80A, # E-80PX, E80AP1, Portland, Oreg.), resistin (R and D Systems, #DRSN00, Minneapolis, Minn.), isoform 1 of fibronectin (Bioxys, #EF1045, Brussels, Belgium), lymphocyte cytosolic protein 1 (LCP1) (Antibodies-online.com, #ABIN415176, Atlanta Ga.), cathespin B (USCNK, #SEC964Hu Houston, Tex.), protein S-100A9/MRP14, biglycan, annexin-A6, (CedarLane #CY-8062, SE9822HU, SE92345HU, Burlington, N.C.) and cellular fibronectin (Biohit #6030010, Helsinki, Finland). Briefly, each assay comprised a single-plex sandwich ELISA with primary antibody specific for the selected protein pre-coated in planar arrays in 96-well microtiter plates. After serum incubation and washing, a second biotinylated antibody to a different site on the protein from the capture epitope was introduced and streptavidin-horseradish peroxidase (HRP) subsequently bound to the biotinylated detection antibody. Chromagen-substrate reagent was added and the absorbance (OD) was read according to manufacturer instructions on a SpectraMax M2e plate reader (Molecular Devices, Sunnyvale, Calif.). The OD values were acquired and processed using a four-parameter curve fit to compare the experimental samples to the recombinant protein calibration curve run in parallel wells to derive absolute protein concentrations adjusted for dilution.

Validation Study of the Selected Serum Biomarkers:

A second stage study of 67 independent serum samples (‘serum validation dataset’) from 36 non-BE GERD and 31 EAC patients from RPCI and AHN was run in order to validate the previous discovery findings from the 32 sample ‘serum discovery dataset’ from the patients. The sample preparation and quality control (QC) methodological protocols and serum ELISAs were performed as before for the ‘serum discovery dataset’ using the final five candidate biomarkers that demonstrated statistical discrimination between the 2 patient groups in the initial dataset.

Development of a Serum Biomarker Panel and Predictive Model for EAC:

Serum ELISA data from the discovery and validation datasets were subsequently used to develop and test predictive biomarker rule models using a new bioinformatics method called Bayesian Rule Learning (BRL) system (Gopalakrishnan et al., Bioinformatics. 2010; 26(5):668-75). The BRL is a set of classification algorithms that was previously successfully applied to biomarker discovery and validation from serum proteomic datasets for early detection of ALS and lung cancer (Ryberg et al., Muscle Nerve. 2010 July; 42(1):104-11; Bigbee et al., J Thoracic Oncol 2012; 7(4): 698-708.).

A rule model consists of a set of IF-THEN rules, as an example:

IF (BGN>245 μg/ml) AND (S100A9>3 ng/ml) AND (MPO>120 ng/ml)
THEN (Class=EAC)

### Posterior Probability=0.917, P=0.0, TP=21, FP=1

This rule states that if a patient sample has biomarkers BGN, S100-A9, and MPO with serum levels greater than 245 μg/ml, 3 ng/ml, and 120 ng/ml, respectively, (defined in the IF part of the rule) then the patient has EAC (defined in the THEN part of the rule). The posterior probability gives the probability of a true positive for EAC, given all positive matches from the rule. The p-value (P) of the rule is obtained from Fisher's exact test. Fisher's exact test (Sokal and Rohlf, Biometry: the principles and practice of statistics in biological research: New York: WH Freeman, 1995) is a significance test appropriate for categorical count data such as number of true positives and number of positives corresponding to each rule.

The BRL system first learns a Bayesian network (BN) (Neapolitan, Learning bayesian networks. Prentice Hall Upper Saddle River, 2004) constrained to the target node (EAC or non-EAC), and subsequently biomarkers are added as potential parents to this node. It learns the BN from the training data and evaluates it using an extension of the K2 score (Gopalakrishnan et al., Bioinformatics 2010; 26(5): 668-75; Cooper and Herskovits, Machine learning 1992; 9(4): 309-47) assuming all models are equally probable a priori (uniform prior distribution). The details of the BRL algorithms are presented in Lustgarten, 2009 (see Gopalakrishnan et al., Bioinformatics 2010; 26(5): 668-75; Cooper and Herskovits, Machine learning 1992; 9(4): 309-47; Lustgarten, A Bayesian Rule Generation Framework for ‘Omic’ Biomedical Data Analysis: University of Pittsburgh, 2009). Since the BRL method can handle only discrete variables, the continuous-valued ELISA data was discretized for each biomarker into a small number of intervals using a method called Efficient Bayesian Discretization (EBD) (Lustgarten et al., BMC bioinformatics 2011; 12(1): 309). For each biomarker, EBD identifies a small number of intervals in the range of values for that biomarker that is optimal in terms of a Bayesian measure (based on the K2 score (Cooper and Herskovits, Machine learning 1992; 9(4): 309-47)). Using EBD to discretize variables has been shown to yield better classification performance on a range of biomedical datasets (Lustgarten et al., BIOCOMP, 2008:527-32).

Predictive rule models were generated from the ‘discovery dataset’ and applied to the ‘validation dataset’, using different values for the user-defined λ parameter, which is the mean of a Poisson distribution that represents the expected number of cut-points between the ranges of continuous values for each biomarker. It was found that, λ values of 0.5 and 1 yielded models with the highest predictive accuracies. In order to use the validation data as a test set for predictive rule models, it was first necessary to normalize the quantities for each biomarker in the discovery and validation datasets together using Equation 1.

$$F = \frac{\frac{1}{{N}_{+} ( {D}_{T} )} {\sum }_{p = 1}^{{N}_{+} ( {D}_{T} )} {D}_{T}^{p}}{\frac{1}{{N}_{-} ( {D}_{V} )} {\sum }_{q = 1}^{{N}_{-} ( {D}_{V} )} {D}_{V}^{q}} \qquad ( 1 )$$

Here, F is the factor of normalization computed for each biomarker, N₊ and N₋ refer to the total number of cases and controls in each of the datasets: training (D_{T}) and test (D_{V}), respectively. The variables p and q iterate over instances with a specific class value (EAC or non-EAC) in the training dataset (p) and validation dataset (q), respectively.

With the normalized dataset values as determined above, predictive rules were generated from the discovery and validation datasets, using each one independently as the training dataset and the test dataset, respectively. The discovery and validation datasets were appended to create a merged dataset to which we then applied ten-fold stratified cross validation. Herein, the combined dataset was randomized and divided into ten almost equal portions. A predictive model was then learned from nine portions of the data, designated as training data, and the remaining set-aside portion was tested. This was done ten times by applying BRL to learn a predictive model over each fold and test that model to obtain performance metrics. Finally, the average accuracy, balanced accuracy (average of sensitivity and specificity) (BACC) and area under the ROC (AUROC) metrics are reported over this ten-fold cross fold validation. To develop the final predictive model BRL was applied to the complete merged dataset.

Sample Size and Statistical Analysis:

The serum validation study required 26 patients per group for an anticipated effect size of 0.8 with a calculated study power of 80% and a target alpha of 0.05. Statistical analyses were performed using SPSS software (IBM, Armonk, N.Y., Version 20). A p-value<0.05 was considered statistically significant.

### Example 2

### Tissue Based Proteomics

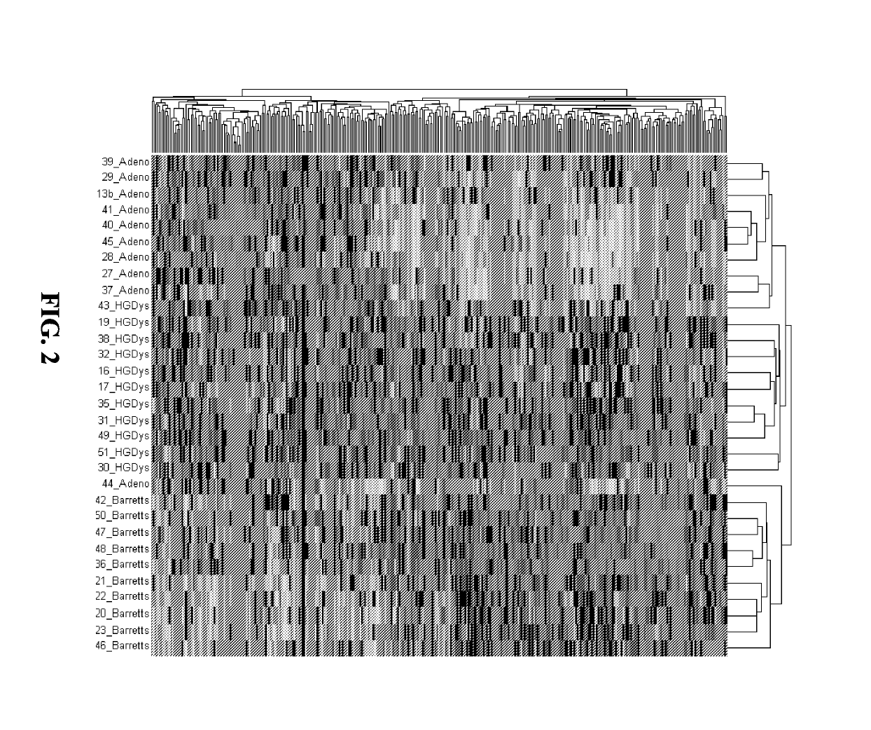

A total of 3,777 proteins were identified from 62 LC-MS/MS analyses (duplicate analyses for each of the 31 tissue samples). The range of total spectral counts obtained in each sample analysis ranged from 2,759 to 5,181 and was significantly associated with the patient groups (Kruskal-Wallis test, p=0.0364). With a minimum threshold of 10 spectral counts, 351 proteins were observed to be differentially abundant along the spectrum of BE, HGD and EAC (Kruskal-Wallis test, p<0.05) (see the table in Example 1 and FIG. 2). These results showed nearly perfect clustering of relative protein abundance from BE to EAC (FIG. 2).

### Example 3

### B-AMP© Biomarker ELISA

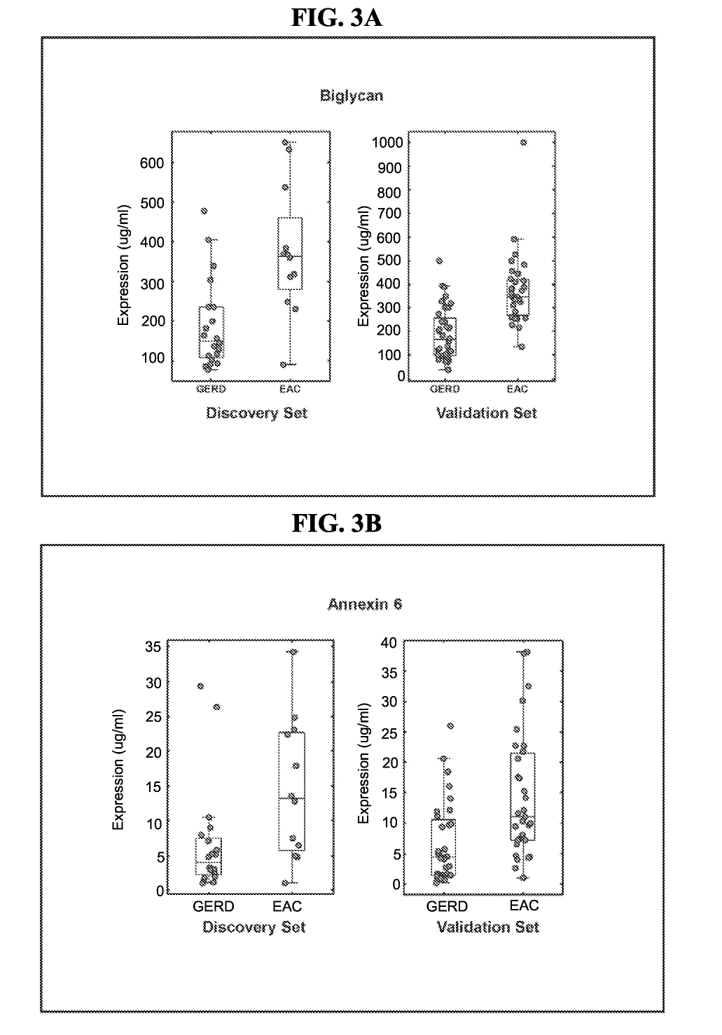

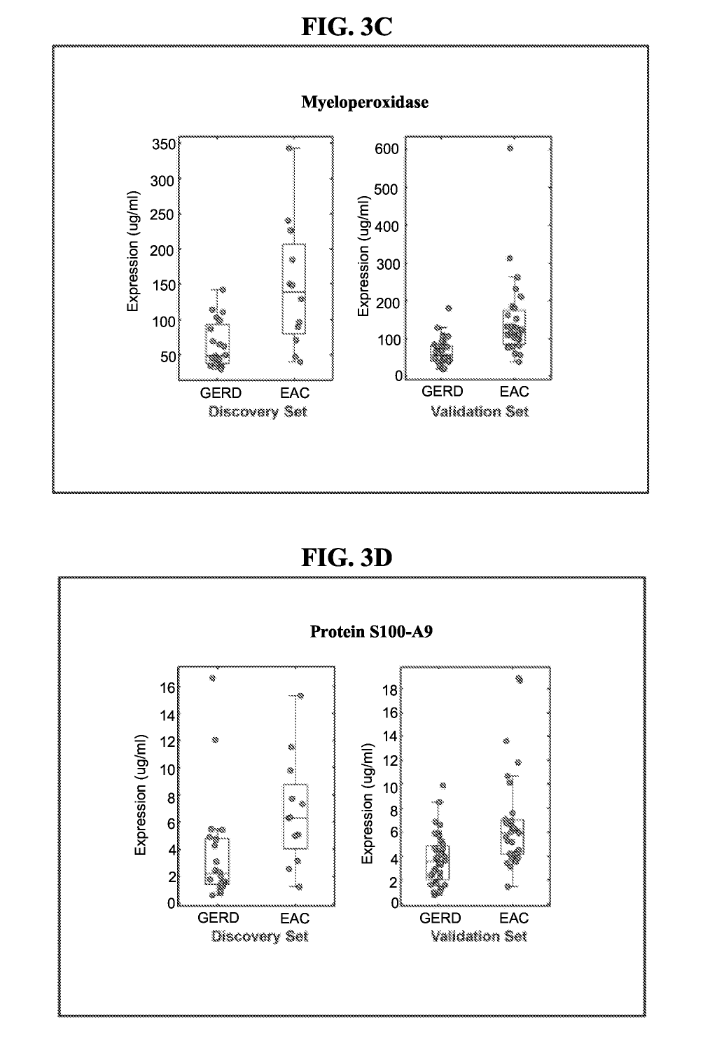

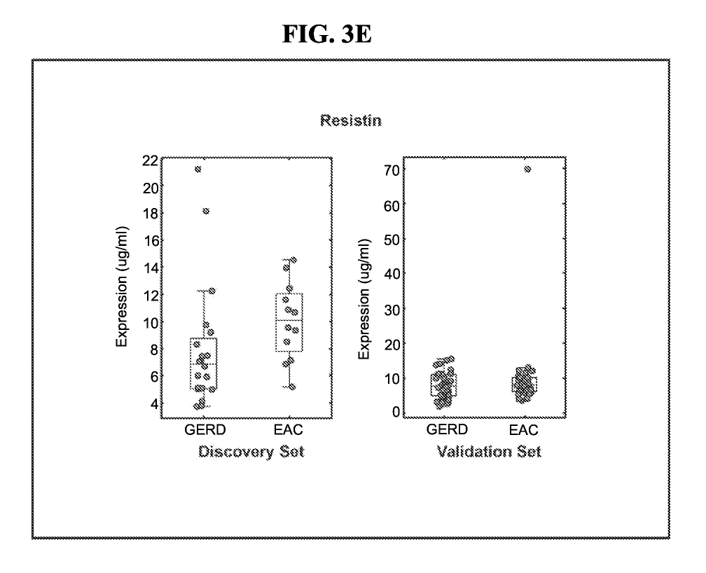

Eleven of these 351 differentially abundant proteins were selected for evaluation in serum using ELISAs on discovery sample sets based on their functional relevance and availability of commercial ELISA kits. The serum ELISA results obtained from these selected tissue-based candidate biomarkers demonstrated significantly elevated serum levels for five of the eleven proteins tested in the samples from EAC patients compared to the samples collected from the non-BE GERD patients in the ‘serum discovery dataset’ (FIG. 3). These included ANXA6, BGN, S100A9, MPO and resistin. The serum levels followed similar trends across the disease spectrum as observed in the corresponding tissue samples (Table 2; FIG. 1).

TABLE 1
Correlation of tissue expression (upregulated proteins represented by the signal
in Group 6 of FIG. 2) determined by LC-MS/MS and spectral counting with serum
abundance by ELISA for the final five candidate protein biomarkers.

| | SERUM RESULTS (UNNORMALIZED MEAN) | | | | | TISSUE RESULTS (MEAN | | | |
|---|---|---|---|---|---|---|---|---|---|
| | | | | pValue | pValue | SPECTRAL COUNTS) | | | |
| PROTEIN | GERD | EAC | Unit | (Ttest) | (Rnk) | BE | HGD | EAC | pValue |
| MYELOPEROXIDASE | 64.36 | 147.23 | ng/ml | 0.00073 | 0.00256 | 0.1 | 3.18 | 7.7 | 0.01257 |
| RESISTIN | 7.93 | 10.05 | ng/ml | 0.15967 | 0.01852 | 0 | 0 | 0.8 | 0.0094 |
| PROTEIN S100-A9 | 3.74 | 6.77 | ng/ml | 0.04708 | 0.00964 | 4 | 6.36 | 12.2 | 0.03244 |
| BIGLYCAN | 190.11 | 375.24 | μg/ml | 0.00065 | 0.00256 | 0 | 0.36 | 1.1 | 0.01151 |
| ANNEXIN-A6 | 6.64 | 14.48 | μg/ml | 0.01956 | 0.02278 | 0.7 | 1.45 | 6.4 | 0.00265 |

Table 1 summarizes the observed differences in candidate biomarker protein abundance measured by LC-MS/MS and spectral counting along the disease spectrum in FFPE-derived tissue samples and their corresponding concentrations in the serum samples determined by ELISA. Largely consistent with the results in the ‘serum discovery set’, the concentrations of all of the biomarkers were significantly higher in the EAC patients' samples in the ‘serum validation set’ with the exception of resistin (FIG. 3).

### Example 4

### Rule Model

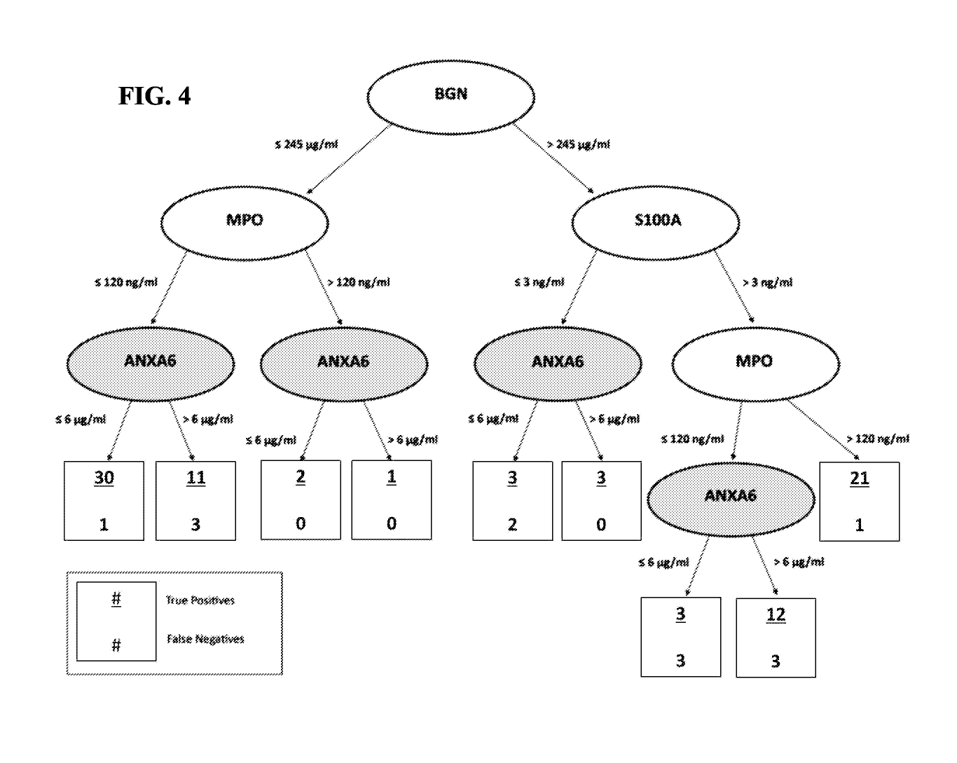

The rule model that was obtained by applying BRL to the merged discovery and validation ELISA datasets is shown visually in FIG. 4. In the tree, the interior nodes (shown as ellipses) represent predictor biomarkers, the leaf nodes (shown as rectangles) show the patient counts for the number of EAC cases and controls respectively, and the labels on the arcs represent the serum biomarker levels. The rules which consist of combinations of individual biomarkers at specific cutoff concentrations produced by BRL are shown below (Table 2). Each rule has a posterior probability associated with it, along with the p-value (P) obtained from Fisher's exact test (Gopalakrishnan et al., Bioinformatics 2010; 26(5): 668-7; Sokal and Rohlf, Biometry: the principles and practice of statistics in biological research: New York: WH Freeman, 1995). Fisher's exact test is applicable in situations wherein the number of samples is fairly small, as in this case, which leads to small numbers of counts for positives covered by a rule. The number of true positives (TP) and false positives (FP) covered by the rule are also presented. This set of rules constitutes the predictive model that can be applied to a future patient for whom these serum biomarker levels have been measured and it is of interest to use the matching rule from among this set of mutually exclusive and exhaustive rules to provide an estimate of the probability that the patient has EAC.

### Example 5

### Evaluation of Alternative Rule Models

The highest accuracy that was obtained when a BRL predictive model was learned using the ‘discovery dataset’ and applied to the ‘validation dataset’ was 76%, with a balanced accuracy (BACC) of 74% and the area under the receiver operating characteristic curve (AUROC) of 86%. The highest accuracy that we obtained when a BRL predictive model was learned using the ‘validation dataset’ and applied to the ‘discovery dataset’ was 75%, with a BACC of 73% and AUROC of 84%.

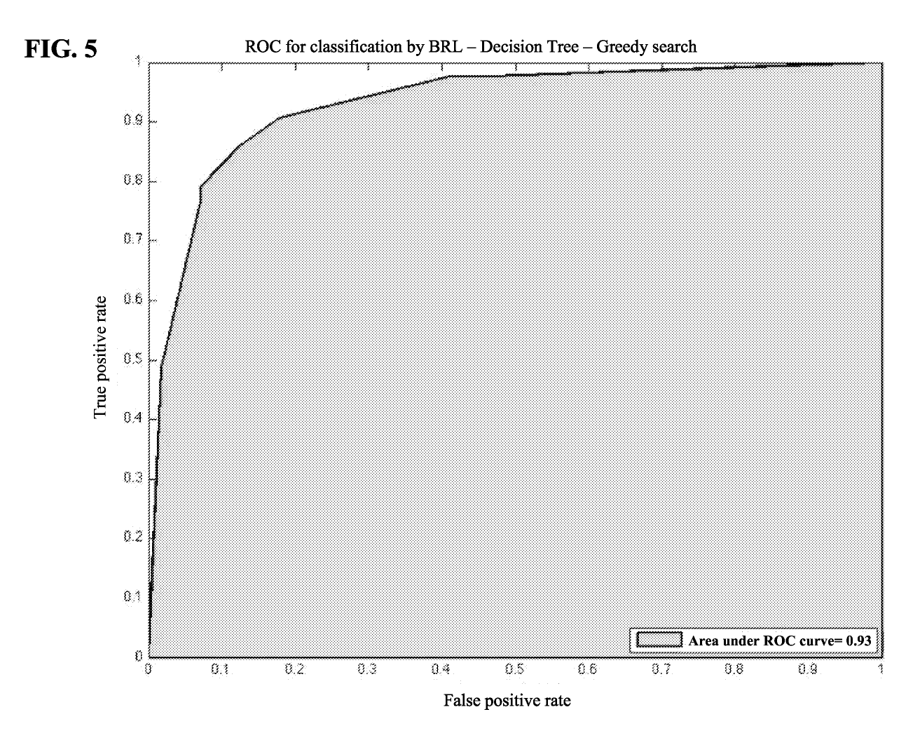

These two reciprocal results indicate that BRL is accurate in modeling the uncertainty in the validity of the rule models because of the similar results obtained in these two independent datasets for EAC classification. The cross-validation results for the merged discovery and validation datasets analyzed by BRL yielded an overall accuracy of 87%, BACC of 86% and AUROC of 93% (FIG. 5).

Serum biomarkers hold significant promise for early non-invasive detection of EAC. However, direct identification of novel biomarkers from serum previously presented a challenging analytical problem due to the very high dynamic range of protein concentrations present in the complex serum proteome (Anderson and Anderson, Mol Cell Proteomics 2002; 1(11): 845-6). This study utilized a unique LC-MS/MS-based tissue proteomics discovery approach which allowed the selection of candidate serum biomarkers that were significantly and differentially abundant in the tissue samples along the disease progression pathway from BE to EAC and have clinical and functional relevance.

As summarized in Table 2 these results demonstrate that the observed differences in abundance in the selected proteins along the BE-HGD-EAC disease spectrum in FFPE-derived tissue samples were mirrored by the corresponding protein biomarker concentrations in the corresponding serum samples. The tissue-based results guided targeted serum based biomarker discovery; and with the ease of serum sample collection and relatively low cost, the disclosed B-AMP© panel and rule model was able to identify patients with EAC with an overall accuracy of 87%. Resistin appeared to not add any predictive value to the best scoring models based on the other four biomarkers.

Over the years, a number of biological, IHC- and transcriptomic tissue-based analyses have been performed toward identification of biomarkers of neoplastic progression of BE. Many reports cite aberrant biological processes that occur in the development of EAC, such as cell cycle abnormalities and numerous genetic and epigenetic alterations, including loss of heterozygosity, polyploidy and aneuploidy. Although proteins such as TP53 (p53) (Ramel et al., Gastroenterology 1992; 102(4 Pt 1): 1220-8; van Dekken et al., Am Jr Clin Pathol 2008; 130(5): 745-53, β-catenin (CTNNB1) (Washington et al., Mod Pathol 1998; 11(9): 805-1), p 16 (van Dekken et al., Am J Clin Pathol 2008; 130(5): 745-53; Barrett et al., Oncogene 1996; 13(9): 1867-73), and cyclin D1 (CCND1) (Bani-Hani et al., J Natl Cancer Inst 2000; 92(16): 1316-21), have been studied as potential tissue-based immunohistochemical biomarkers of progression (Ong et al., World J Gastroenterol 2010; 16(45): 5669-81), none have resulted in widespread clinical adoption or demonstrated adequate clinical utility likely due to the genetic heterogeneity of EAC between patients. Thus, a single biomarker has not been demonstrated to be of use for determining progression of EAC.

With respect to the biomarkers identified in the disclosed panel, biglycan (BGN) (an extracellular matrix component with a known role in epithelial-to-mesenchymal transdifferentiation central to BE carcinogenesis), annexin-A6 (ANXA6) (belonging to the annexin family of calcium and phospholipid binding proteins that is a motility promoting factor), myeloperoxidase (MPO) (an oxidant generating enzyme linked to cancer progression) and protein S100A9 (that promotes tumor growth in inflammation associated cancer development) individually have prognostic significance in several tumor types including esophageal squamous cell carcinoma (Castillo-Tong et al., Tumour Biol 2014; 35(1): 141-8; Fan et al., J Proteomics 2012; 75(13): 3977-86; Zhu et al., Int J Clin Exp Pathol 2013; 6(11): 2497-505; Lomnytska et al., Br J Cancer 2011; 104(1): 110-9; Sakwe et al., Exp Cell Res 2011; 317(6): 823-37).

Previous studies on serum protein biomarkers of EAC, were limited to an early report of elevated levels of the squamous cell carcinoma antigen (SCC), carcinoembryonic antigen (CEA), and cytokeratin 19-fragment (CYFRA 21-1) in advanced esophageal cancer patients (Kawaguch et al., Cancer 2000; 89(7): 1413-7) and more recent evaluations of the circulating lymphocyte antigen 6 complex locus K (LY6K) and elevated serum levels of serum gastrin in patients with a diagnosis of HGD or EAC (Ishikawa et al., Cancer Res 2007; 67(24): 11601-11; Wang et al., Am J Gastroenterol 2010; 105(5): 1039-45). A2007 comparative mass spectrometry proteomics analysis identified candidate tissue proteins in surgical specimens that by hierarchical clustering analysis accurately discriminated BE and EAC and identified 38 differentially abundant proteins among which Rho GDP dissociation inhibitor 2, α-enolase, lamin A/C, elongation factor Tu, thioredoxin domain-containing protein 17 and nucleoside-diphosphate kinase A that were upregulated in both mRNA and protein expression in EAC compared to BE (Zhao et al., Mol Cell Proteomics 2007; 6(6): 987-99). Several of the these proteins or their isoforms along with previously reported progression related proteins were also differentially abundant in our tissue discovery dataset, these included CTNNB1, Rho GDP dissociation inhibitor 2, elongation factor Tu and thioredoxin domain-containing protein 17.

The disclosed tissue-based studies have been extended to demonstrate the feasibility of using LCM and LC-MS/MS analysis of esophageal tissue biopsy specimens for robust proteomic analysis (Stingl et al., J Proteome Res 2011; 10(1): 288-98). The disclosed study demonstrated the ability of rule learning methods to successfully predict class values accurately using two independent datasets with similar distributions of cases and controls. Serum-based proteomic biomarker panels and risk prediction models can be extended to determine the presence or absence of BE, HGD or EAC. Advantages include: (1) improved clinical resource utilization using blood-based detection as a means of directing effective screening and post-therapy surveillance; (2) detect progression from BE to HGD or EAC in patients undergoing surveillance; (3) prevent death from EAC by employing an assay in high-risk patients to identify BE, HGD and EAC; (4) track therapeutic response and thereby enable tailored therapy based on individual disease biology; and (5) detect subclinical EAC recurrence prior to the development of recurrence as detected by clinical imaging.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of detecting the likelihood that a subject will develop esophageal adenocarcinoma, comprising:
detecting a level of biglycan, annexin-A6, myeloperoxidase and protein S100-A9 in a biological sample from the subject; and
comparing the level of biglycan, annexin-A6, myeloperoxidase and protein S100-A9 to a respective control level of biglycan, myeloperoxidase, and protein S100-A9 polypeptide, or a nucleic acid encoding the protein.

2. (canceled)

3. A method of diagnosing Barrett's esophagus, low grade dysplasia of the esophagus, or high grade dysplasia of the esophagus, and performing esophageal endoscopy in a subject, comprising:
obtaining a biological sample from the subject, wherein the biological sample is an esophageal tissue sample, a blood sample, a plasma sample or a serum sample,
detecting a level of biglycan, annexin-A6, myeloperoxidase, and protein S100-A9 in the biological sample from the subject; and
comparing the level of biglycan, annexin-A6, myeloperoxidase, and protein S100-A9 to a respective control level of biglycan, annexin-A6, myeloperoxidase, and protein S100-A9;
diagnosing the subject with Barrett's esophagus, low grade dysplasia of the esophagus, or high grade dysplasia of the esophagus, when the level of biglycan, annexin-A6, myeloperoxidase, and protein S100-A9 is detected in the biological sample is increased as compared to a respective control; and
performing esophageal endoscopy on the diagnosed subject.

4. The method of claim 3, wherein the control is a standard value of biglycan, myeloperoxidase, annexin-A6 and protein S100-A9, respectively, in one or more subjects known not to have esophageal adenocarcinoma.

5. (canceled)

6. The method of claim 3, wherein the sample comprises a blood sample, a plasma sample or a serum sample.

7. The method of claim 1, further comprising assessing the clinical risk factors for the subject.

8. The method of claim 7, wherein the clinical risk factors comprise one or more of gastric esophageal reflux disease (GERD), smoking, alcohol use, and Barrett esophagus.

9. The method of claim 3, wherein detecting biglycan, annexin-A6, myeloperoxidase, and protein S100-A9 comprises detecting biglycan, annexin-A6, myeloperoxidase and protein S100-A9 polypeptide.

10. The method of claim 9, wherein detecting biglycan, annexin-A6, myeloperoxidase and protein S100-A9 polypeptide comprises performing mass spectrometry.

11. The method of claim 10, wherein the mass spectrometry is MALDI-TOF mass spectrometry and/or LC-mass spectrometry.

12. The method of claim 9, wherein detecting biglycan, annexin-A6, myeloperoxidase, and/or protein S100-A9 polypeptide comprises contacting the biological sample or a component thereof with an antibody that specifically binds biglycan, an antibody that specifically binds myeloperoxidase, an antibody that specifically binds protein S100-A9, and/or an antibody that specifically binds annexin-A6 protein.

13. The method of claim 12, wherein detecting biglycan, annexin-A6, myeloperoxidase, and/or protein S100-A9 polypeptide comprises performing an immunoassay.

14. The method of claim 13, wherein the immunoassay is a Western blot, an enzyme linked immunosorbent assay, or a radioimmunoassay.

15. The method of claim 12, wherein the antibody that specifically binds biglycan, the antibody that specifically binds myeloperoxidase, the antibody that specifically binds protein S100-A9, and/or the antibody that specifically binds annexin-A6 is directly labeled.

16. The method of claim 15, wherein the label is a radioactive marker, a fluorescent marker, an enzyme or a metal.

17. The method of claim 3, wherein detecting biglycan, annexin-A6, myeloperoxidase, and protein S100-A9 comprises detecting biglycan, annexin-A6, myeloperoxidase and protein S100-A9 mRNA.

18. The method of claim 17, wherein detecting biglycan, annexin-A6, myeloperoxidase and protein S100-A9 mRNA comprises performing polymerase chain reaction, a microarray analysis or a hybridization reaction.

19. The method of claim 18, wherein the polymerase chain reaction is reverse transcriptase polymerase chain reaction (RT-PCR).

20. The method of claim 1, further comprising administering to the subject a therapeutically effective amount of an agent for the treatment or prevention of esophageal adenocarcinoma if the subject is determined to have an increased likelihood of developing esophageal adenocarcinoma.

21. The method of claim 20, wherein the agent is surgical treatment, an antibody, radiation, a pharmaceutical agent, laser therapy or electrocoagulation.

22. The method of claim 3, wherein the subject is diagnosed with Barrett's esophagus.

23. The method of claim 1, wherein the method stratifies the risk of developing esophageal adenocarcinoma.

24. (canceled)

25. The method of claim 21, wherein the agent is an antibody that specifically binds epithelial growth factor receptor, an antibody that specifically binds HER2, or an antibody that specifically binds vascular endothelial growth factor.

26. The method of claim 25, wherein the agent is cetuximab, trastuzumab, or bevacizumab.

27. The method of claim 21, wherein the surgical treatment is endoscopic submucosal surgical dissection (ESD), minimally invasive esophageal (MIE) surgery, endoscopic mucosal resection (EMR), cryoablation, or radiofequency ablation (RFA).
